(19) United States
(12) Patent Application Publication
(10) Pub. No.: US 2009/0214614 A1
(43) Pub. Date: Aug. 27, 2009

(54) Method for Cell Implantation

(75) Inventors: Hanne Everland, Bagsvaerd (DK); Christian Clausen, Fredensborg (DK); Kurt Osther, Scottsdale, AR (US)

Correspondence Address: JACOBSON HOLMAN PLLC, 400 SEVENTH STREET N.W., SUITE 600, WASHINGTON, DC, 20004, US

(73) Assignee: INTERFACE BIOTECH A/S, Horsholm (DK)

(21) Appl. No.: 11/991,312

(22) PCT Filed: Sep. 5, 2006
(86) PCT No.: PCT/US2006/034673

§ 371 Date: Mar. 20, 2009

Related U.S. Application Data
(60) Provisional application No. 60/713,645, filed on Sep. 2, 2005.

(51) Int. Cl. A61K 9/00; A61P 43/00

(52) U.S. Cl. 424/423

(57) ABSTRACT

The present invention provides for a method for tissue engineering by cell implantation that involves the use of a scaffold in situ at the site of a defect, where the therapeutic cells are fixed in place into the scaffold only once the scaffold is inserted at the site of the tissue defect, thereby locking not only the cells to the scaffold, but also the scaffold to the tissue defect. The invention also provides a kit of parts suitable for performing the method of the invention.

### FIELD OF THE INVENTION

The present invention relates to the implantation of living cells into a tissue defect in a living mammal in order to promote repair of the tissue defect.

### BACKGROUND OF THE INVENTION

Tissue engineering methods using cell transplantation are known, and for example, may involve for instance open joint surgery (e.g., open knee surgery) and, in case of joint surgery, extensive periods of relative disability for the patient to recuperate in order to ensure that optimal results are achieved. Such procedures are costly, and require extensive medical procedures such as rehabilitation and physical therapy.

Methods using scaffold technologies of various forms, where the scaffold (with, or without cells grown in the scaffold) is inserted into the defect, have suffered from difficulties in performing the cell implantation procedure solely guided by arthroscopy.

Arthroscopic Autologous Cell Implantation (called AACI or ACI using minor surgical Interventions) is a surgical procedure for treating cartilage or bone defects, whereby a scaffold is inserted into the defect concomitantly with applying cell suspension or cell mixture with precursor fixatives, into said defect using a needle as for instance a “blunt” needle or a catheter. This implantation procedure is visualized and guided by an arthroscope.

WO 2004/110512 discloses an endoscopic method, useful for treating cartilage or bone defects in mammals, involving identifying the position of defect and applying chondrocytes, chondroblasts, osteocytes and osteoblasts cells into cartilage or bone defect. The cells are applied with a solidaflable support material, such as soluble thrombin and fibrinogen or collagen mixtures. It is envisaged that, for surgery in a convex or concave joint, that a porous membrane may be placed at the site of defect, but removed once the fibrin/cell mix are coagulated in place. The method disclosed in WO 2004/110512 allows tissues to be repaired arthroscopically, i.e. without the need of open joint surgery (e.g., open knee surgery).

However, the method disclosed in WO 2004/110512 do not provide a complete or sufficiently robust repair of the defects, particularly in cases where patients over-stress the sites of surgery before sufficient repair/regeneration has occurred.

Scaffolds are porous structures into which cells may be incorporated. They are usually made up of biocompatible, biodegradable materials and are added to tissue to guide the organization, growth and differentiation of cells in the process of forming functional tissue. The materials used can be either of natural or synthetic origin.

Previously filed Danish priority application PA200600337 (In the name of Coloplast) provides preferred scaffold materials for use in the methods and kit of parts of the present invention. The scaffold materials, and methods for the production of the scaffold materials are not part of the present invention.

The present invention provides an improved method for performing tissue engineering by cell transplantation, incorporating a solid scaffold Into the site of the defect, and concomitantly applying the cells and a fixative precursor (such as the ‘solidifiable support’ materials disclosed in WO 2004/110512.).

The conversion of the fixative precursor to a fixative results in effective attachment of both the cells to the scaffold, and the scaffold to the site of defect. We have surprisingly discovered that methods according to the present invention provide remarkable rates of tissue healing, and recovery to an extent which can resemble surrounding undamaged tissue. The effect of the scaffold not only increases the cell migration (e.g. a chrondogenic effect) and viability, but also provides a robust support for the cells, allowing both the generation of structurally robust tissue, and also allows a more uniform integration of the engineered or replacement tissue to the surrounding tissue.

### BRIEF DESCRIPTION OF FIGURES

FIG. 1. Histology (A) Toluldine Blue Staining on 10 μm cryosectlon. (B) Safranin O staining of a 10 μm cryosectlon.

FIG. 2. Immunohistochemistry (A) Collagen type II observed as brown to dark spots. (B) Aggrecan observed as brown to dark spots.

FIG. 3. Gene Expression. SOX9 and Collagen type II is showing enhanced expression, in the Hydrogel-PolyGraft System, which has also been observed by us, when applying the hACs to our Hydrogel (SCAS principle) (Fibrinogen/thrombin composition with chondrocytes e.g., with other types of scaffolds encompassed in this invention).

FIG. 4. Scaffolds were placed on the bottom of a 12 well NUNC tray; cells/fibrin was added to the scaffolds in rows labelled #5 and #18 in triplicate. The last two (2) triplicate set-ups contain phosphate buffered saline (PBS) only.

FIG. 5. Application of chondrocytes/hydrogel on the bottom of the cartilage defect.

FIG. 6. The MPEG-PLGA has now been placed into the scaffold.

FIG. 7. Macroscopic ICRS score. The SCAS System (PLGA on the histogram) scores significant higher compared to the three other groups.

FIG. 8. Representative histological appearance of repair tissue if the defects were left untreated or they were treated with microfracture. As demonstrated very little repair tissue is observed. Safranin O staining and toluidine blue staining.

FIG. 9. Representative histological appearance of repair tissue if the defects were treated with FIB50 or treated with the SCAS System. As demonstrated, high degree of fill is observed with the SCAS System. (SCAS principle). Safranin O staining and toluidine blue staining were used.

FIG. 10. Migration of chondrocytes out of a cartilage explant, placed In MPEG-PLGA/hydrogel scaffold after 2 weeks.

FIG. 11. Migration of chondrocytes out of a cartilage explant, placed In MPEG-PLGA/hydrogel scaffold after 4 weeks.

FIG. 12. Overview of a method of the invention. An individual with a defect (D) in a tissue (T) is previously identified (1), a scaffold (S) is prepared and inserted Into the defect (2) and concomitantly application of the cell suspension and fixative precursor (CF), and a suitable conversion agent for conversion of the fixative precursor to a fixative (T), to the scaffold in situ in the defect, is performed.

FIG. 13. Examples of two suitable kit of parts, shown in use during the methods of the invention. In the first kit of parts (1), the first component scaffold (S) is impregnated with the conversion agent (T) (i.e. (ST)), the cell suspension (C) comprising the fixative precursor (F) (i.e.(CF)) is supplied in separate container which is connected to a delivery means (X) to the site of defect (D) In the tissue (T). The (CF) component may be stored in two separate containers, and joined by a common connector/mixing device (Y) which is linked to the delivery means (X) (this embodiment is not shown). Application of pressure to the delivery device (P) forces (CF) out of the container, through to the delivery device, and directly onto the scaffold at the site of the defect. In the second kit of parts (2), the first component scaffold (S) is not impregnated with the conversion agent (T), however (T) is concomitantly mixed with the (CF) component by the pressure applied (P), which forces the (CF) and (T) out of their respective containers and Into a common connector/mixing device (Y), the mixture of (CF) and (T) is concomitantly applied via a delivery device (X), to the previously implanted scaffold (S) at the defect (D) In the tissue (T).

FIG. 14. Examples of a third suitable kit of parts, shown in use during the methods of the invention. In the kit of parts shown, the first component scaffold (S) is not impregnated with the conversion agent (T), the cell suspension (C), fixative precursor (F), and conversion agent (X) are supplied in separate containers which are joined to a common delivery means (X) to the site of defect (D) in the tissue (T). Application of pressure to the delivery device (P) forces (C), (F) and (X) out of their respective containers, through to a common connector/mixing device (Y), and then the mixture of (C), (F) and (X) is concomitantly applied, via a delivery device (X), to the previously implanted scaffold (S) at the defect (D) In the tissue (T).

FIG. 15. Shows examples of suitable connection devices which may be used in the kit of parts according to the Invention. (F) refers to the join between the connection device and the fixative precursor source/container, (C) refers to the join between the connection device and the cell suspension source/container ((F) and (C) may be a single source), (T) refers to the join between the connection device and the conversion agent source/container. (Y) refers to the connection device body, (X) refers to the delivery device, and an optional flexible tube between the delivery device and the connection device, (M) refers to an optional mixing component of the connection device which facilitates the mixing of the components (F), (C) and (T) to form (F, C and T).

### SUMMARY OF THE INVENTION

It has been surprisingly found in the present invention that the use of a scaffold and fixed cells, such as cells and scaffold fixed in a gel or hydrogel, provides a highly efficient and robust method of creating new healthy tissue at the site of a defect, which remarkably has been shown to be capable of resulting in remarkably high quality repairs, which are thicker, more uniform and in some cases almost seamless with the original undamaged adjacent tissue. For example, the present method can be used to efficiently prepare high quality ‘hyalln’ like articular cartilage, typically a type of tissue which takes a long time to re-grow after surgery, and often results in weak and inferior repairs. In some aspects the repairs performed by the present invention are considered almost equivalent to, or are approaching the appearance of, natural cartilage.

In a first aspect the Invention provides for a method for repairing defects in a tissue of a living individual mammal, such as a human being, by transplantation of living mammalian cells, said method comprising:
i) The concomitant application of
a) A first component comprising an essentially cell-free, biocompatible scaffold;
b) a second component comprising a mixture of essentially serum free mammalian cells and a biologically acceptable fixative precursor (or a mixture of precursors),
   to the site of said defect, prior to the conversion of the fixative precursor(s) into a fixative, and
ii) fixing said mammalian cells to said scaffold, and said scaffold to said living mammalian tissue by conversion of the fixative precursor into a fixative.

In a second aspect the Invention provides for a kit of parts, for use in the above method, said kit comprising
a) A first component comprising an essentially cell-free, biocompatible scaffold;
b) a second component comprising a mixture of essentially serum free mammalian cells and a biologically acceptable fixative precursor (or a mixture of precursors),
c) and optionally a cross-linking agent for said fixative precursor wherein, said first component and said second component are isolated from one another.

The embodiments described herein which refer to the components used in the method of the invention equally apply to the kit of parts according to the invention.

### DETAILED DESCRIPTION OF THE INVENTION

The object of the novel methods and means for applying a scaffold of any tolerated type, included but not limited to collagen scaffolds, alginate, polylactic acid (PLA), polyglycolic acid (PGA) compositions and compositions of above described scaffold or membrane—like scaffolds—onto a target, such as for instance a cartilage defect, bone defect, skin, and organ defects or localized cell defects in organs.

The scaffold, without cells or the fixative (such as adhesive/glue), is preferably, as a first step placed at the site of the defect, typically after having been cut or “sized” to fit the defect—suitably the scaffold may be molded to a particular shape or form to suit the site of defect and/or the desired shape/form of the new tissue.

In one aspect, the cells mixed with culture medium and fibrinogen are placed on the surface of the cell-free scaffold previously placed in the defect—the cells and fibrinogen, being applied onto the surface of the cell-free scaffold located in the target area, and the component containing the gelating catalyst/agent such as the thrombin, is added on top of the scaffold either simultaneously or shortly thereafter. The cells and fibrinogen, and the thrombin, are absorbed through the hydrophilic scaffold. The gelating or clotting process takes place within seconds or minutes (such as less than 5 minutes, or even less than 2 minutes) in the scaffold thus locking the cells to the scaffold at the same time as locking the scaffold (and In one aspect cells) to the defect.

A preferred aspect of the present invention is where a scaffold, such as a cell-free or essentially cell free scaffold, is placed in the defect prior to the addition of cells. Suitably, the scaffold forms a tight continuous fit along the area of the defect to be treated. The scaffold should preferably have the ability of being hydrophilic.

In another aspect, the cells in one fixative precursor (e.g., fibrinogen is mixed with the other fixative precursor (thrombin) concomitantly with the insertion of the scaffold.

In yet another aspect of the invention, the scaffold may be prepared in such a manner that it, prior to use, is “impregnated” with one fixative precursor, which is capable of retaining its activity (e.g., the thrombin analogues developed by HumaGene Inc., Chicago, Ill.). The scaffold is typically cut or shaped into the size of the defect, the scaffold is then placed in the defect (for instance during arthroscopic guidance), the cells, mixed with the fixative precursor, or precursors, is placed on the scaffold, and the cell/fixative precursor will then contain a solution, which when added to the scaffold, impregnated with the other fixative precursor (e.g., thrombin analogue), will render the fixative precursor already in the scaffold active, thereby enabling it to react with the fixative precursor added together with the cells, resulting in gelation, clotting and adhesion.

The methods described are named the Scaffold Hydrophilic Cell Absorption system (the SCAS system). The SCAS system may be applied using any cells on any suitable target ranging from cartilage defects, osteoarthritic defects, bone defects, periodontal defects, skin defects, as well as various “target” organs to which cells would benefit the patient for repairing a disorder in said organs. For Instance a scaffold covering an area of damaged skin, needing autologous dermal transplantation, could be placed on the area needing transplantation, the cells in medium containing one of the gelating or coagulating components (e.g., fibrinogen) could be brought together with the other gelating or coagulating component(s) and absorbed, when sprayed over the hydrophilic membrane for instance due to the fact that the membrane will quickly absorb the cells in medium containing fibrinogen and at the same time absorbing the gelating factor, as for instance thrombin, thus keeping the membrane sticking to the damaged skin area—the entire procedure typically taking place in a sterile or aseptic heap-filtered tent (LAF-bench) or room. The cells, such as fibroblasts and/or skin cells, such as keratinocytes or other skin related cells preferably of autologous origin will be locked in the scaffold or membrane and at the same time the scaffold or membrane will adhere to the target due to the in situ gelating or coagulating cell containing composition applied onto the cell-free scaffold or membrane when said cell-free scaffold or membrane is placed in its target (e.g., cartilage defect).

In one embodiment, after the placement of the scaffold, the cell suspension, for instance suspended in culture medium containing fibrinogen, and another solution containing a clotting agent, for example thrombin, along with possible other clotting agents, are placed using, for instance the kit of parts as disclosed herein, which may comprise at least two syringes, which may be functionally or structurally connected, so as to release, for instance, one or more drops of this combined components or components containing both cell suspension and clotting agent mixture (e.g. fibrinogen and thrombin), onto the scaffold. A hydrophilic scaffold then facilitates a “suction” of the combined cell fluid and clotting agent into the scaffold, thereby locking and adhering the scaffold, now filled with cells, included in the fibrin dispersed in the scaffold, at the target site (defect) as for instance in the cartilage defect. Therefore the scaffold has been filled with the cells and at the same time clotting agents are interacting in the scaffold, which is therefore kept in place instantly (such as with a few seconds or minutes of application of the combined cell fluid and clotting agent into the scaffold).

In previous disclosures made by K. Osther and others (U.S. Pat. Nos. 5,759,190; 5,989,269; 6,120,514; 6,283,980; 6,379,367; 6,592,598; 6,592,599; 6,599,300; 6,599,301), the cells are applied in the scaffold and cultured into the scaffold for some time prior to placing the cell containing scaffold in the target (e.g., cartilage defect). The present invention results in improved results and a more convenient procedure, as described herein.

In the method of the present invention, the scaffold is not removed from the site of defect as part of the same surgical procedure. Preferably, the scaffold is not removed, although may biodegrade or dissolve at the site of defect, typically over a period of at least 1-6 months after surgery. Therefore during and subsequent to the method of the invention, the scaffold remains at the site of defect, although may blodegrade or dissolve at the site of defect, typically over a period of at least 1-6 months after surgery.

The cells may be prepared as described in WO02/061052, which is hereby incorporated by reference. The cells disclosed in WO02/061052 may therefore be injected directly into a cell-free scaffold placed on its target.

In one embodiment, cells are locked into the scaffold due to the cell fluid and gelating material being added simultaneously or essentially concurrently, to the cell-free scaffold (or membrane) already placed in the target area. The cell containing gelating substance applied into the cell-free scaffold (or membrane) are therefore dispersed simultaneously or essentially concurrently, with the gelating substance which is also applied as a fluid to the cell-free membrane. In one embodiment, the cells are locked into the scaffold by the formation of a fibrin gel (hydrogel), by the interaction of the gelating materials fibrinogen with thrombin. The scaffold then adheres spontaneously to the target area (e.g., the cartilage defect).

### EMBODIMENTS OF THE INVENTION

It is preferable that the first component is applied to site of said defect, prior to application of said second component. In such an embodiment it is considered possible that the first component may be applied in a separate surgical procedure than the subsequent application of the second component, for example the first component may be applied using an open surgical procedure, whilst the second surgical procedure may be endoscopically applied. However, it is preferred that the first and second components are applied during the same surgical procedure.

Once the first and second components have been applied, and fixed, the mammalian cells are allowed to migrate and/or grow through the scaffold to generate new living tissue, this may, in the case of cartilage repair, be referred to as a chondrogenic effect

In a preferred embodiment of the invention a fixative precursor conversion agent is concomitantly applied to site of said defect.

In a preferred embodiment, the conversion agent is applied as part of said first component. In this respect the conversion agent may be incorporated into the scaffold such as in the form of particles or chemically linked or non-chemically linked. The conversion agent may be applied as a solvent, which for instance is allowed to dry in the scaffold, and/or, for example be covalently linked to the scaffold polymer, or alternatively chemically or radiation cross linked to the polymer or associated by ionic attraction (e.g. hydrogen bonding or van der vaals force).

In one embodiment, the conversion agent may be a cross-linking agent and/or a polymerization agent.

In one embodiment, the conversion agent is a protein or a polysaccharide.

In one embodiment, the conversion agent is lyophilized with said biologically acceptable scaffold.

In a preferred embodiment, the fixative is in the form of a hydrogel, ie. a gelating substance capable of binding water, for example fibrin formed by the combination of the fixative precursor fibrinogen and the conversion agent thrombin.

It is recognized that, whilst it is preferable that the fixative precursor is part of the second component, and that the conversion agent is supplied separately, either as part of the first component, or mixed concomitantly with the cells upon application to the scaffold, it is possible that in one specific embodiment, the conversion agent may be mixed with the cells (for example thrombin) and the conversion agent, such as fibrinogen is provided either concomitantly, or as part of the first component, for example as a freeze dried component of the scaffold. In this specific embodiment, for the purposes of the invention, the conversion agent and the fixative precursor are interchangeable terms, what is important is that prior to the method of the invention, that they are kept separate, therefore allowing concomitant, or essentially simultaneous, application during the method of the invention.

In a preferred embodiment, the mammalian cells are immuno-compatible with said living mammalian tissue/individual. The use of non immuno-compatible cells may however be used, for example with immuno surpressive drugs.

### The Patient

The living individual mammal is preferably a human being, typically a patient. However the methods of the invention may also be applicable to other mammals, such as horse or a goat.

### The Cells

It is preferable that said mammalian cells are obtained or derived from said individual mammal. Such methods of obtaining and culturing cells from the individual mammal are disclosed in WO02/061052.

The mammalian cells are supplied preferably in the form of a cell suspension or tissue explant. Tissue explants may be directly taken from other parts of the Individual mammal, and may therefore be in the form of tissue grafts such as a skin graft.

The mammalian cells may be autologous, homologus (allogenic) or xenogenic in origin.

The mammalian cells may originate from multipotent or pluripotent stem cells.

The mammalian cells may be selected from the group consisting of: fibroblasts, keratinocytes, chondrocytes, endothelial cells, chondrocytes, osteoblasts, neural and periodontal cells. Cells of mesenchymal origin.

Fibroblasts and keratinocytes are two cell types of skin cells.

Chondrocytes are particularly preferred, such as for cartilage repair.

It is envisaged that stem cells, or other suitable precursor cells which are capable of becoming or producing chondrocytes once in situ at the site of the defect may also be used.

Typically, the cells used in the second component are present in a sufficient amount of cells to result in regeneration or repair of the target tissue or defect, such as of about 0.1×10⁴ to about 10×10⁶ cells/ml, or 0.1×10⁶ cells/ml to about 10×10⁶ cells/ml.

### The Tissue Defect

Whilst it is recognized that the method of the Invention will be widely applicable to a large number of solid tissues in the mammalian body, it is preferred that the tissue defect is selected from the group consisting of: cartilage defect, bone defect, skin defect, periodontal defect.

### Surgical Method

The surgical method may be performed as, or during a method of surgery, such as a method of endoscopic, atheroscopic, or minimal invasive surgery, or conventional or open surgery.

### The Scaffold

It is highly preferred that the scaffold is hydrophilic and/or is prepared prior to insertion onto the site of defect by the application of a biocompatible wetting agent, for example a hydrogel.

It is also highly preferred that the scaffold is porous to water and/or an isotonic buffer

In one embodiment, the scaffold essentially consists or comprises, such as comprise a majority of, a polymer, or polymers, of molecular weight, such as average molecule weight, greater than about 1 kDa, such as between about 1 kDa and about 1 million kDa, such as between 25 kDa and 75 kDa.

The scaffold may be in the form selected from the group consisting of: a membrane, woven or non woven fibers, freeze dried polymer such as freeze dried polymer sheets, rods or tubes.

In one preferred embodiment the scaffold is synthetic.

The scaffold may be in a form selected from the group consisting of; a sheet, a membrane, a molded form, a plug, a tube, a sphere, a three dimensional form prepared for Insertion into site of defect, or an implant.

The method of the invention may be used for cosmetic reconstruction—for example, the scaffold is made/molded into the shape required for reconstructive surgery, and the cells applied or fixed to the scaffold once in situ.

The scaffold may be pre-molded to fit the exact shape of the defect, either by using the defect as a mound, or by creating the defect in a mold which is prepared using the defect as a template.

The first component first, such as the scaffold, or compositions/compounds added to the scaffold, e.g. during freeze drying, may comprise further compound, which may for example enhance cell adhesion, cell migration and/or tissue regeneration, these may, for example be selected from the group consisting of; hyaluronic acid (HA), hydroxyl apatite (e.g. In the form of granules), growth factors, such as IGF-1, and collagen.

Indeed the pores of the scaffold may be partly occupied by a component which facilitates the cell adhesion and/or in-growth for regeneration of tissue, such as a component selected from the group consisting of: Chondroitin sulfate, hyaluronan, heparin sulfate, heparan sulfate, dermatan sulfate, growth factors, fibrin, fibronectin, elastin, collagen, gelatin, and aggrecan.

It is also envisaged that in one embodiment the second component, may comprise such compounds, which may for example enhance cell migration and/or tissue regeneration.

In one interesting embodiment, the amount of compounds which enhance cell migration and/or tissue regeneration, such as hyaluronic acid, is incorporated Into the scaffold, such as at a proportion of between about 0.1 and about 15 wt %, such as between 0.1 and 10 wt %, such as such as between 0.1 and 10 wt %. In one embodiment the level Is below 15 wt %, such as below 10 wt % or below 5 wt %. In one embodiment the level is above 0.01 wt % such as above 0.1 wt %, or above 1 wt %.

The scaffold may consists or comprises of any suitable biologically acceptable material, however in a preferred embodiment the scaffold comprises of a compound selected from the group consisting of: poly lactide (PLA), polycaprolacttone (PCL), polyglycolide (PGA), poly(D,L-lactide-co-glycolide) (PLGA), MPEG-PLGA (methoxypolyethyleneglycol) poly(D,L-lactide-co-glycolide), poly hydroxyacids in general. In this respect the scaffold, excluding the pore space and any additional components, such as those which facilitates the cell adhesion and/or in-growth for regeneration of tissue, may comprise at least 50%, such as at least 60%, at least 70%, at least 80% or at least 90%, of one or more of the polymers provided herein, including mixtures of polymers.

PLGA and MPEG-PLGA are particularly preferred.

The scaffold may be prepared by freeze drying a solution comprising the compound, such as those listed above, in solution.

It is preferred that the scaffold has a porosity in the range of 20% to 99%, such as 50 to 95%, or 75% to 95%.

In one embodiment the scaffold comprises a biological polymer, such as protein or polysaccharide. Suitable biological polymers may be selected from the group consisting of: gelatin, collagen, alginate, chitin, chitosan, keratin, silk, cellulose and derivatives thereof, and agarose.

Biologically Acceptable Fixative Precursor

In one embodiment, the biologically acceptable fixative precursor is a biologically obtained or derived component, such as fibrinogen.

The fibrinogen may be in the form of recombinant fibrinogen (e.g., recombinant human fibrinogen from HumaGene Inc., Chicago, Ill., USA)

The fibrinogen may be Isolated from a mammalian host cell such as a host cell obtained or derived from the same species as the individual mammal, or a transgenic host.

Suitable concentrations of fibrinogen used Include 1-100 mg/ml.

In one embodiment, particularly when the fixative precursor is fibrinogen, the conversion agent may be selected from the group consisting of: thrombin, a thrombin analogue, recombinant thrombin or a recombinant thrombin analogue.

Suitable concentrations of thrombin used is between 0.1NIH unit and 150NIH units, and/or a suitable level of thrombin for polymerizing 1-100 mg/ml fibrinogen.

Standard NIH units refers to the routinely used National Institute of Health standard unit for measurement of Thrombin, which according to Gaffney P J, Edgell (Thromb Haemost. 1995 September; 74(3):900-3, is equivalent to between 1.1 to 1.3 IU, preferably 1.15 IU, of thrombin.

The living mammalian tissue may be, for example selected from the group consisting of: connective tissue, skin, cartilage, bone, ligaments, and periodontal tissue.

In one embodiment, the method is performed during reconstruction surgery or cosmetic surgery.

### Kit of Parts

The kit of parts, suitable for use in the above methods, comprise a first component and a second component as defined according to the method of the invention or elsewhere herein, wherein, said first component and said second component are isolated from one another. It is therefore imperative that prior to performing the surgical procedure, the first and second components are not combined.

Suitably, it is preferred that a conversion agent as defined herein is also provided and wherein conversion agent is isolated from said first and said second component.

The conversion agent may, as described herein be part of said first component.

The structural form of the kit of parts may take any form which allows the first and second components, and optionally the conversion agent, to be kept separate prior to performing the surgical procedure, but combined during the surgical procedure so that the scaffold is inserted into the defect either concomitantly, or prior to the addition of the second component, or in one embodiment, subsequent to the addition of the second component, as described herein in reference to the method of the invention.

Preferably, the kit of parts comprises an integrated supply device, comprising the following functionally linked devices: (i) at least one container which contains said second component prior to use, (ii) a force applicator for pressurizing said second component out of said container and into (iii) a (mixing) connector to (iv) a delivery device, wherein said delivery device is suitable for direct application of the second component to the first component inserted in the site of defect in living mammalian tissue.

In one embodiment, the integrated supply device comprises two containers, a first container which contains said cell suspension, and a second container which contains said fixative precursor, wherein said first and second containers are joined by a common connector to allow mixing of said cell suspension and said fixative precursor concomitantly to prepare the second component prior to delivery by said delivery means.

As explained herein, it is also practiced that the cell suspension and the fixative precursor are mixed prior to use, therefore it is not always necessary to have two separate containers for each in the integrated supply device.

The integrated supply device may, however, comprise a further container which contains said conversion agent, wherein said further container is joined by a common connector to said first, and optionally said second containers by said common connector to allow mixing of said first component with said conversion agent immediately prior to delivery by said delivery means to said first component inserted in the site of defect in said tissue. Such a integrated supply device may also comprise the embodiment described above referring to the separate containers for cell suspension and fixative precursor.

The containers may be functionally linked to a force applicator which may be either a common force applicator device or separate independent force applicators. The force applicators may be structurally linked, for example by linking or joining the proximal ends of syringe plungers, or they may be linked, for example by computer coordinated peristaltic pumps.

Although the container may take on any suitable form, such as tubes, cylinders, bags, jars, beakers etc., in one embodiment at least one container, such as the first container, the second container and/or said third container, are in the form a syringe body, and said force applicator(s) are in the form of the respective syringe plungers, which may or may not be joined.

The connector typically is, or comprises, at least one tube, which has a proximal end/or ends which is/are connected to said one or more containers, as referred to above, and a single distal end which is connected to said delivery device.

The connector may further comprises a mixing device to allow thorough mixing of the second component and optionally said conversion agent prior to entry into said delivery device. The mixing device may be in the form, for example of a simple propeller like shape, which creates mixing vortices in the flow of liquid, ensuring uniform mixing. The mixing device may be externally powered, e.g. by an electric motor, or a passive device which rotates using the flow of liquids past the surface of the propeller. Alternatively the connector may act as a static mixer.

The delivery device, may be in the form of any suitable medical device for the selected surgery technique, such as those referred to herein, for example the delivery device may be selected from the group consisting of: a catheter, a needle, a syringe, a tube, a pressure gun, and a spraying device.

Whilst the above unified connector and delivery device is preferred, it is recognized that the kit of parts may comprise parallel integrated supply device, for example one for the second component, and one for the conversion agent.

### FURTHER ASPECTS OF THE INVENTION

The first component comprises or consists of a solid scaffold.

The second component comprises the cells (mammalian cells) and a biologically acceptable fixative precursor. Typically the second component is in the form of a cell suspension.

In one preferred embodiment, the first composition is stored dry or in a humidified condition, but is wetted prior to use, such as in a saline or isotonic buffer, or the appropriate buffer system.

The term “concomitant application” as used herein refers to the application to form a mixture of the first component and the second component, and suitably a conversion agent which converts the fixative precursor to the fixative, so that the mixture is present at the site of defect prior to the fixation step (process). In the method according to the invention, it is highly preferred that the scaffold (i.e. the first component,) is added to the site of defect prior to the addition of the second component. Addition of the second component prior to the first component may result in a liquid gap between the scaffold and the defect, resulting in a poor fixation of the scaffold to the defect, and a higher likelihood that the scaffold, and cells enclosed within fall to fix effectively to the defect.

However, in one embodiment the first component (scaffold) is placed in the site of defect subsequent to the application of the second component. In such an embodiment, the scaffold is placed prior to the fixation step. The conversion agent may therefore have (very recently) been applied, e.g. as part of the second component, or may be applied either as part of the first component, or shortly after the application of the first component. In this embodiment whether the application of the second component precedes the application of the first component, the time between the application of the second and first components is, preferably, no greater than 30 minutes, such as no greater than 10 minutes, no greater than 5 minutes, no greater than 2 minutes, such as no greater than 1 minute.

The conversion agent may be provided as part of the first component, for example prepared with the scaffold, or may be added to the second component either immediately prior to, during or subsequent to the application of the second component to the first component.

The term “Blocompatible” refers to a composition or compound, which, when inserted into the body of a mammal, such as the body of patient, particularly when inserted at the site of the defect does not lead to significant toxicity or a detrimental immune response from the individual.

### The Scaffold

A “Biocompatible scaffold” refers to a solid scaffold that is tolerated when inserted into the body of a mammal, such as the body of patient, particularly when inserted at the site of the defect. The scaffold is not removed as part of the method of the invention/surgical procedure, but may blodecompose (bio-degrade) (or reabsorb) over time, such as between 1-6 months after surgery, such as once the defect has been suitably repaired by the migration and growth of the cells throughout the scaffold and suitably together with the surrounding tissue. The time for biodecomposition may vary significantly between different applications.

In one embodiment, the (biocompatible) scaffold preferably comprises a polymer, which may be selected from the group consisting of: collagen, alginate, polylactic acid (PLA), polyglycolic acid (PGA), MPEG-PLGA or PLGA.

The scaffold may be in a multiple of different forms, such as a form selected from the group consisting of: a porous membrane, a porous sheet, an implant, a fibre, a three dimensional shape, such as a mushroom shape, a foam, a tube, Woven or non woven sheet, a rod or, any combinations of these.

Suitably, scaffolds may be of any type and size, as well as any thickness of a scaffold, such as ranging from thin membranes to several millimetres thick scaffolds. In one embodiment, it is also considered that liquid scaffold matrixes may be used, i.e. cell free or essentially cell free scaffolds that form a solid scaffold once placed in situ in the defect, but prior to addition of the cells. However, it is preferred that the scaffolds are solid prior to addition to the defect.

The scaffold, or first component, is preferably cell free, or essentially cell free, prior to use in the methods of the invention.

The required type of scaffolds used within the context of this invention shall be scaffolds that do not act as foreign bodies in the mammal (including humans) so that no immunity or a minimum of immunity may be observed and the scaffolds used in this context shall not be toxic or significantly harmful to the organism in which it is placed. Preferably, the scaffold does not contain any microbial cells, or any other harmful contaminants. Cells used in the scaffold for instance human cells embedded in a hydrogel, shall be capable of being placed onto the scaffold, after said scaffold is placed in its target area. The scaffold should preferably be hydrophilic so that the cell material relatively quickly is absorbed into the scaffold. However, in some instances, scaffolds may be accessible by injection with the cells and hydrogel. The cells should tolerate the scaffold with no toxic or only a minimal degree of toxicity, or no significant toxicity which may otherwise lead to detrimental results.

In a preferable embodiment, the scaffold is in the form of a sheet, which may be precut or sized to fit the defect. Such a scaffold may be, for example between 0.2 mm to 6 mm thick.

In a highly preferred embodiment, the scaffold is hydrophilic, i.e. has the ability to absorb at least a small amount of water or aqueous solution (such as the cell suspension composition, e.g. the hydrogel solution), such as absorb at least 1%, such as at least such as at least 2%, such as at least 5%, such as at least 10%, such as at least 20%, such as at least 30%, such as at least 50% of the scaffold volume, of water (or equivalent aqueous solution) when placed in an aqueous solution, such as a physiological media, a buffer, or water, it is particularly beneficial that the scaffold can absorb the above amounts of the cell suspension into its porous structure, thereby providing a relatively homogenous distribution of cells throughout the scaffold once inserted and fixed into the site of defect.

The term hydrophilic is used interchangeably with the term ‘polar’.

In the case when a non-polar scaffold is used, it is preferable that the scaffold is pretreated with an agent which facilitates the update of cells, such as a wetting agent. Wetting agents may also be used in conjunction with hydrophilic scaffolds to further improve cell penetration into the porous structure.

The biocompatible scaffold of the invention may comprise or consist of a polyester. By incorporation of a hydrophilic block in the polymer, the biocompatibility of the polymer may be improved as it improves the wetting characteristics of the material and initial cell adhesion is impaired on non-polar materials.

In a preferred embodiment the scaffold is biodegradable, i.e. will, over a period of time degrade inside the mammalian body, such as between 1-6 months.

It is highly preferred that the scaffold is porous, e.g. has a porosity of at least 25%, 50%, such as in the range of 50-99.5%. Porosity may be measured by any method known in the art, such as comparing the volume of pores compared to the volume of solid scaffold. This may be done by determining the density of the scaffold as compared to a non-porous sample of the same composition as the scaffold. Alternatively Mercury Intrusion Porosimetry may be used.

In a highly interesting embodiment of the invention, the biocompatible scaffold according to the invention consists or comprises of one or more of the polymers selected form the group comprising: poly(L-lactic acid) (PLLA), poly(D/L-lactic acid) (PDLLA), Poly(carprolactone) (PCL) and poly(lactic-co-glycolic acid) (PLGA), and derivatives thereof, particularly derivatives which comprise the respective polymer backbone, with the addition of substituent groups or compositions which enhance the hydrophilic nature of the polymer e.g. MPEG or PEG. Examples are provided herein, and include a highly preferred group of polymers, MPEG-PLGA

The term “Essentially cell free”, when used in reference to the first component, refers to that the scaffold does not comprise the living mammalian cells prior to use in the method according to the invention. In one embodiment, the term “essential cell free” is equivalent to “cell free”, and means that the scaffold is sterile, and comprises no living micro-organism or mammalian cells which could survive and/or replicate once introduced into the patient, preferably no living cells whatsoever.

In one embodiment, the scaffold consists or comprises a synthetic polymer.

### The “Defect”

The term “Defect” as used herein refers to any detrimental or injured condition of a tissue, which is associated with existing, or future, loss of, or hindered function, disability, discomfort or pain. The defect is preferably associated with a loss of normal tissue, such as a pronounced loss of normal tissue. It is envisaged that the methods of the invention may be used prophylactically, i.e. to prevent the occurrence of defects, or for preventing the deterioration of an existing defect. The defect may, for example be a cavity in the tissue, a tear or wound in the tissue, loss of tissue density, development of aberrant cell types, or caused by the surgical removal of non-healthy or injured tissue etc. In a preferred embodiment, the defect could either an Injured articular cartilage, an articular cartilage defect down to and/or involving the bone (osteoarthritis), a combination of cartilage and bone defect, a defect in bone which is surrounded by normal cartilage or bone, or a defect in a bone structure itself or be a bone structure that needs re-inforcement by addition of bone cells with scaffold as in the SCAS system. In a most preferred embodiment, the defect is in cartilage, such as articular cartilage or a skin defect.

### The “Tissue”

The term “Tissue” as used herein refers to a solid living tissue which is part of a living mammalian individual, such as a human being. The tissue may be a soft tissue (e.g. internal organs an skin) or a hard tissue (e.g. bone, joints and cartilage). The tissue may be selected from the group consisting of: cartilage, such as articular cartilage, bone, skin, teeth, ligament, and tendon, or any other mesenchymal tissue.

### The “Cells”

The term “living mammalian cells”, which may also be referred to as “mammalian cells” or “cells” herein, refers to cells that are obtained from or derived from cells obtained from a mammalian tissue, which have been maintained or cultured in vitro, preferably in a suitable culture medium, prior to use in the method according to the invention. In one preferred embodiment, the term “living mammalian cells” refers to chondrocytes, chondroblasts, osteocytes and osteoblasts, periodontal cells, or cells derived from skin and/or combinations thereof. In a preferred embodiment, the cells are obtained from or derived from the living individual mammal, i.e. are autologous. The cells may also be homologous, i.e. compatible with the tissue to which they are applied, or may be derived from multipotent or even pluripotent stem cells, for instance in the form of allogenic cells. In one embodiment the cells may be allogenic, from another similar individual, or xenogenic, i.e. derived from an organism other than the organism being treated. The allogenic cells could be differentiated cells, progenitor cells, or cells whether originated from multipotent (e.g., embryonic or combination of embryonic and adult specialist cell or cells, pluripotent stemcells (derived from umbilical cord blood, adult stemcells, etc.), engineered cells either by exchange, insertion or addition of genes from other cells or gene constructs, the use of transfer of the nucleus of differentiated cells into embryonic stemcells or multipotent stem cells, e.g., stem cells derived from umbilical blood cells.

Therefore in one embodiment, the method of the invention also encompasses the use of stem cells, and cells derived from stem cells, the cells may be, preferably obtained from the same species as the individual mammal being treated, such as human stem cells, or cells derived there from.

In a preferred embodiment, particularly for repair of cartilage, bone and/or skin, the cells are mesenchymal cells, chondrogenic cells or cells derived from skin.

### Fixative/Fixative Precursor

The fixative precursor used in the invention may be any form of biocompatible glue or adhesive, including gelation agents, which are capable of being absorbed by the porous scaffold and, when converted into the fixative capable of anchoring both the scaffold to the defect and the cells to the scaffold and optionally the cells also to the defect.

WO 2004/110512 provides several fixative precursors, which are referred to as ‘support materials’—i.e. those materials which are capable of coagulating or solidifying upon application to the defect.

Suitable fixative precursors may be a protein such as a protein selected form the group consisting of: fibrinogen, gelatin, collagen, collagen peptides (type I, type II and type III),

The fixative precursor may be a polysaccharide such as agarose or aiginase.

Suitably, the fixative may be a biocompatible medical adhesive.

It is preferable that the fixative precursor is biocompatible, and may for example be human proteins which have either been obtained from humans, or alternatively recombinantly expressed. Human fibrinogen is a preferred fixative precursor, polymerizing for instance when exposed to for instance thrombin.

A preferred fixative is fibrin.

The fixative precursor may be a gelation agent, which, when suitably converted to a fixative forms a gel structure around and through the scaffold, thereby fixing the scaffold and cells.

WO2004/110512, which is hereby incorporated by reference, provides specific examples of suitable combinations of fixative precursors and conversion agents. Suitably, the ratio of fixative precursor to conversion agent may be used to control both the rate at which the fixation occurs, and the level of support the fixed composition provides to the cells.

In a preferred embodiment the conversion of the fixative precursor to the fixative occurs via the application of a conversion agent. The addition of the conversion agent preferably occurs once the scaffold is in site of the defect. The addition of the conversion agent to the second component, preferably occurs immediately prior to, simultaneous to, or immediately after the addition of the second component to the scaffold—i.e. the effect of the conversion agent in converting the fixative precursor to a fixative, such as a gel/hydrogel or solid, occurs only once the scaffold and cells are in situ at the site of the defect, and typically the cells have been distributed through the scaffold.

In one embodiment, such as when the fixative precursor is fibrinogen, the conversion agent is thrombin or a thrombin analogue. Factor XIII, sodium, calcium or magnesium ions may be added to facilitate the conversion, either to the conversion agent or first or second component. In a specific embodiment, ions, or salts such as sodium, calcium magnesium, etc. that may facilitate the thrombin cleavage effect on fibrinogen rendering a polymerization may be added. Thrombin of any origin may be used, although it is preferable that a biologically compatible form is used—e.g. human recombinant thrombin may be used in the treatment of human tissue defects. Alternatively other sources of thrombin may be used, such as bovine thrombin, however bovine thrombin may induce immune reactions in for instance humans.

Once the first and second components are in situ in at the site of the defect the components are fixed in place by conversion of the fixative precursor(s) to the fixative. Although it is envisioned that this, in some embodiments, not require a conversion agent, it is recognized that a conversion agent provides a highly controllable and convenient method of ensuring uniform and effective fixation of both scaffold and cells.

The conversion agent thrombin may be incorporated into the scaffold and the hydrogel will be formed when adding the fibrinogen/cell suspension to the scaffold.

Fixation may take the form of forming a gel (i.e. gelation) such as a hydrogel which locks the cells into the scaffold, and the scaffold into the defect, whilst allowing a suitable medium for cell migration and growth, thereby facilitating the growth of new tissue through the scaffold and repairing the defect.

### Preparation of the Cells

WO02/061052, hereby incorporated by reference, provides suitable methods for preparing the cells for use in the present invention.

Prior to use, the living mammalian cells are typically placed in a suitable suspension with a culture media, which may optionally comprise growth hormones, growth-factors, adhesion-promoting agents, and/or physiologically acceptable ions, such as calcium and/or magnesium ions (see WO 2004/110512). It is highly preferably that the cell suspension does not comprise significant levels of blood serum, i.e. are essentially serum free, such as free of autologous or homologous blood serum, particularly if the serum contains components which may interfere with the formation of the fixative in situ at the defect site. One example is the use of serum that comprises thrombin, which if added to a first component which comprises fibrinogen, may accelerate the formation of the fixative prior to the placement at the site of defect. In one embodiment, the use of small amounts of serum or the addition of inert stabilizing serum proteins may therefore be acceptable, if they do not interfere with the method according to the invention.

Prior to use, the cell suspension may be kept together with the medium alone, and thereafter mixed with the fixative precursor to form the second component.

In one embodiment, the cell suspension may be mixed with the fixative precursor, prior to, simultaneously or even immediately after application of the fixative precursor to the scaffold—i.e. the second component may be formed in situ. However, it is preferred that the second component, comprising both the mammalian cells and fixative precursor are combined prior to application to the site of defect, and/or said first component.

### Kit of Parts

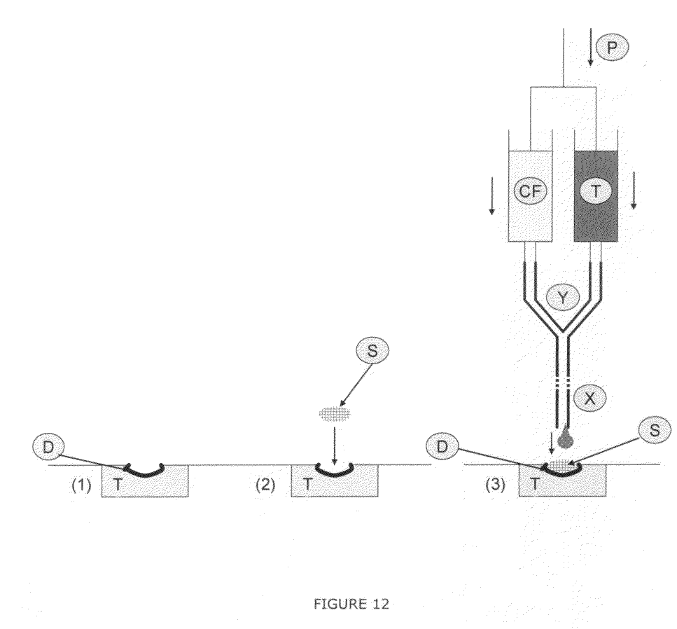

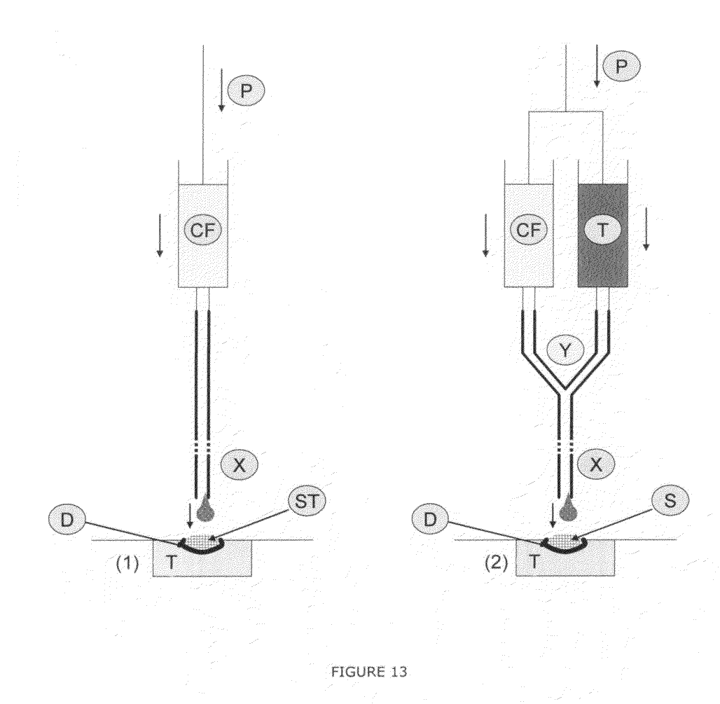

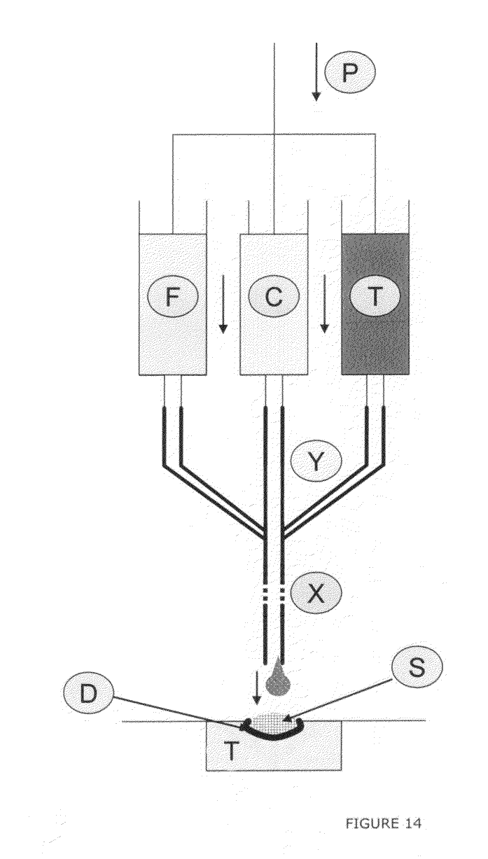

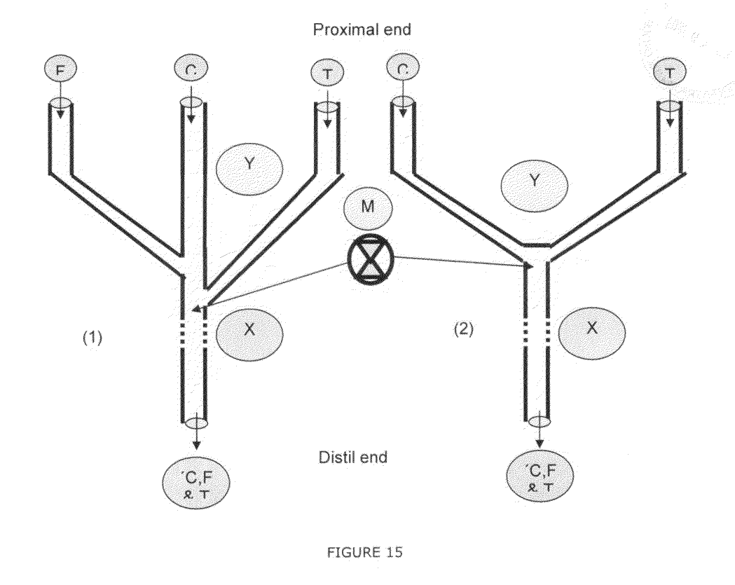

FIGS. 12-15 provide diagrammatic representation of suitable kits of the Invention, their employment in the method of the invention (FIG. 12) and connector devices which form part of the devices shown.

In a kit of parts according to the invention, the second component, comprising the mammalian cells are suitably located together with the fixative precursor in a container, such as one chamber of a delivery device. However, it is also envisaged that the fixative precursor may be present in a separate chamber of the same or functionally associated with a second container (such as a chamber or delivery device), i.e. which is functionally connected to said first chamber to allow the formation of the second component either immediately before or even during application of the second component to the site of defect. It is however preferred that the cells and the fixative precursor are combined as a unified second component prior to the method of the invention.

It is important that the fixative precursor does not form the fixative (e.g. form a gel and clot the cells) in the chamber itself, but only after being located into the scaffold at the site of defect. The conversion agent, when used, is therefore preferably not added to the second conversion agent until either immediately before, during or even subsequent to the application of the second component to the scaffold at the site of defect. The kit of parts may therefore comprise a further container which comprises said conversion agent, which may be functionally connected to said first chamber (an optionally said second chamber) to allow the formation of a mixture of said second component and said conversion agent, either immediately before or even during application of the second component to the scaffold at the site of defect. It is also envisaged that the conversion agent may be applied subsequent to the concomitant application of said first and second components to site of defect, however, this is may, in some circumstances, lead to un-uniform fixation and as a result a lower quality result.

In one embodiment, the kit of parts of the present invention comprises a first component (scaffold), and either a twin or triple chambered delivery device, such as a syringe, where the chambers are functionally connected to allow mixing of the contents of the two/three chambers during application. The second component (comprising cells and fixative precursor) is present in either one chamber, or in the cells suspension and fixative precursor are present in two separate chambers, and the conversion agent is present in a third chamber. The chambers may, for instance, be functionally connected via a “Y” connection to one single connection, so that when the contents of the chambers are released, e.g. in the case of syringes by applying pressure to the syringe plungers (which may also structurally or functionally associated, e.g. by being fused together at the plunger ends), the first component and the conversion agent are combined. Suitably the act of mixing the contents of the two/three chambers, results in the concomitant application of the first component/conversion agent mixture onto the scaffold at the site of defect—as way of illustration, the first component/conversion agent mixture is formed when passing through an injection needle or catheter to the scaffold at the site of defect.

In the embodiment where the conversion agent is encompassed in the first component, it is not necessary to mix the conversion agent and the second component prior to application, although it may optionally be combined with this embodiment. Typically, however, the kit of parts relating to this embodiment comprises the first component (scaffold), and a container, such as a delivery device, which contains said second component. However, as described above, the second component may be formed by two separate chambers, one containing the cell (suspension) and the other the fixative precursor (solution).

### Preferred Polymers Used in the Preparation of the Scaffold

DK application PA200600337 discloses methods for the preparation of such preferred polymers for use as scaffolds in the present invention. The following disclosure referring to the preferred polymers for use in the preparation of the scaffold, including the methods for the preparation of such polymers has been disclosed previously in DK application PA200600337.

Preferred biodegradable polymers for use in the method of the invention are composed of a polyalkylene glycol residue and one or two poly(lactic-co-glycolic acid) residue(s).

Hence, in one aspect of the for use in the method of the present invention the scaffold is prepared from, or comprises or consists of a polymer of the general formula:

A-O—(CHR¹CHR²O)ₙ—B

wherein

A is a poly(lactide-co-glycolide) residue of a molecular weight of at least 4000 g/mol, the molar ratio of (i) lactide units and (ii) glycolide units in the poly(lactide-co-glycolide) residue being in the range of 80:20 to 10:90, in particular 70:30 to 10:90,

B is either a poly(lactide-co-glycolide) residue as defined for A or Is selected from the group consisting of hydrogen, C₁₋₆-alkyl and hydroxy protecting groups,

one of R¹ and R² within each —(CHR¹CHR²O)— unit is selected from hydrogen and methyl, and the other of R¹ and R² within the same —(CHR¹CHR²O)— unit is hydrogen,

n represents the average number of —(CHR¹CHR²O)— units within a polymer chain and is an integer in the range of 10-1000, in particular 16-250,

the molar ratio of (III) polyalkylene glycol units —(CHR¹CHR²O)— to the combined amount of (i) lactide units and (II) glycolide units In the poly(lactide-co-glycolide) residue(s) is at the most 20:80,

and wherein the molecular weight of the copolymer is at least 10,000 g/mol, preferably at least 15,000 g/mol, or even at least 20,000 g/mol.

Hence, the polymers for use in the method of the invention can either be of the diblock-type or of the triblock-type.

The porosity of the polymer is preferably at least 50%, such as in the range of 50-99.5%.

It is understood that the polymer for use in the method of the invention comprises either one or two residues A, i.e. poly(lactide-co-glycolide) residue(s). It is found that such residues should have a molecular weight of at least 4000 g/mol, more particularly at least 5000 g/mol, or even at least 8000 g/mol.

The poly(lactide-co-glycolide) of the polymer can be degraded under physiological conditions, e.g. in bodily fluids and in tissue. However, due to the molecular weight of these residues (and the other requirements set forth herein), it is believed that the degradation will be sufficiently slow so that materials and objects made from the polymer can fulfil their purpose before the polymer is fully degraded.

The expression “poly(lactide-co-glycolide)” encompasses a number of polymer variants, e.g. poly(random-lactide-co-glycolide), poly(DL-lactide-co-glycolide), poly(mesolactide-co-glycolide), poly(L-lactide-co-glycolide), poly(L-lactide-co-glycolide), the sequence of lactide/glycolide in the PLGA can be either random, tapered or as blocks and the lactide can be either L-lactide, DL-lactide or D-lactide.

Preferably, the poly(lactide-co-glycolide) is a poly(random-lactide-co-glycolide) or poly(tapered-lactide-co-glycolide).

Another important feature is the fact that the molar ratio of (i) lactide units and (ii) glycolide units in the poly(lactide-co-glycolide) residue(s) should be in the range of 80:20 to 10:90, in particular 70:30 to 10:90.

It has generally been observed that the best results are obtained for polymers wherein the molar ratio of (i) lactide units and (ii) glycolide units in the poly(lactide-co-glycolide) residue(s) is 70:20 or less, however fairly good results were also observed when for polymer having a respective molar ratio of up to 80:20 as long as the molar ratio of (iii) polyalkylene glycol units —(CHR¹CHR²O)— to the combined amount of (i) lactide units and (ii) glycolide units in the poly(lactide-co-glycolide) residue(s) was at the most 8:92.

As mentioned above, B is either a poly(lactide-co-glycolide) residue as defined for A or Is selected from the group consisting of hydrogen, C1 6-alkyl and hydroxy protecting groups.

In one embodiment, B Is a poly(lactide-co-glycolide) residue as defined for A, i.e. the polymer is of the triblock-type.

In another embodiment, B is selected from the group consisting of hydrogen, C1 6-alkyl and hydroxy protecting groups, i.e. the polymer is of the diblock-type.

Most typically (within this embodiment), B is C₁₋₆-alkyl, e.g. methyl, ethyl, 1-propyl, 2-propyl, 1-butyl, tert-butyl, 1-pentyl, etc., most preferably methyl. In the event where B is hydrogen, i.e. corresponding to a terminal OH group, the polymer is typically prepared using a hydroxy protecting group as B. “Hydroxy protecting groups” are groups that can be removed after the synthesis of the polymer by e.g. hydrogenolysis, hydrolysis or other suitable means without destroying the polymer, thus leaving a free hydroxyl group on the PEG-part, see, e.g. textbooks describing state-in-the-art procedures such as those described by Greene, T. W. and Wuts, P. G. M. (Protecting Groups in Organic Synthesis, third or later editions). Particularly useful examples hereof are benzyl, tetrahydropyranyl, methoxymethyl, and benzyloxycarbonyl. Such hydroxy protecting groups may be removed in order to obtain a polymer wherein B is hydrogen.

One of R¹ and R² within each —(CHR¹CHR²O)— unit is selected from hydrogen and methyl, and the other of R¹ and R² within the same —(CHR¹CHR²O)— unit is hydrogen. Hence, the —(CHR¹CHR²O)ₙ— residue may either be a polyethylene glycol, a polypropylene glycol, or a poly(ethylene glycol-co-propylene glycol). Preferably, the —(CHR¹CHR²O)ₙ— residue is a polyethylene glycol, i.e. both of R¹ and R² within each unit are hydrogen.

n represents the average number of —(CHR¹CHR²O)— units within a polymer chain and is an integer in the range of 10-1000, in particular 16-250. It should be understood that n represents the average of —(CHR¹CHR²O)— units within a pool of polymer molecules. This will be obvious for the person skilled in the art. The molecular weight of the polyalkylene glycol residue (—(CHR¹CHR²O)ₙ—) is typically in the range of 750-10,000 g/mol, e.g. 750-5,000 g/mol.

The —(CHR¹CHR²O)ₙ— residue is typically not degraded under physiological conditions, by may—on the other hand—be secreted in vivo, e.g. In from the human body.

The molar ratio of (iii) polyalkylene glycol units —(CHR¹CHR²O)— to the combined amount of (i) lactide units and (ii) glycolide units in the poly(lactide-co-glycolide) residue(s) also plays a certain role and should be at the most 20:80. More typically, the ratio is at the most 18:82, such as at the most 16:84, preferably at the most 14:86, or at the most 12:88, In particular at the most 10:90, or even at the most 8:92. Often, the ratio is in the range of 0.5:99.5 to 18:82, such as in the range of 1:99 to 16:84, preferably in the range of 1:99 to 14:86, or in the range of 1:99 to 12:88, in particular in the range of 2:98 to 10:90, or even in the range of 2:98 to 8:92.

It is believed that the molecular weight of the copolymer is not particularly relevant as long as it is at least 10,000 g/mol. Preferably, however, the molecular weight is at least 15,000 g/mol. The “molecular weight” is to be construed as the number average molecular weight of the polymer, because the skilled person will appreciate that the molecular weight of polymer molecules within a pool of polymer molecules will be represented by values distributed around the average value, e.g. represented by a Gaussian distribution. More typically, the molecular weight is in the range of 10,000-1,000,000 g/mol, such as 15,000-250,000 g/mol. or 20,000-200,000 g/mol. Particularly interesting polymers are found to be those having a molecular weight of at least 20,000 g/mol, such as at least 30,000 g/mol.

The polymer structure may be illustrated as follows (where R is selected from hydrogen, C1-6-alkyl and hydroxy protecting groups; n is as defined above, and m, p and ran are selected so that the above-mentioned provisions for the poly(lactide-co-glycolide) residue(s) are fulfilled):

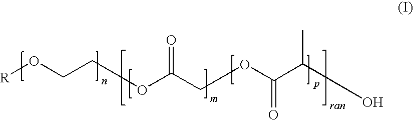

diblock-type polymer

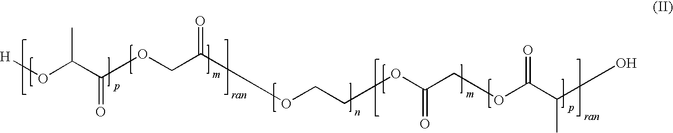

triblock-type polymer

For each of the above-mentioned polymer structures (I) and (II) will be appreciated that the lactide and glycolide units represented by p and m may be randomly distributed depending on the starting materials and the reaction conditions.

Also, it is appreciated that the lactide units may be either D/L or L or D, typically D/L or L.

As mentioned above, the poly(lactide-co-glycolide) residue(s), i.e. the polyester residue(s), is/are degraded hydrolytically in physiological environments, and the polyalkylene glycol residue is secreted from, e.g., the mammalian body. The biodegradability can be assessed as outlined in the Experimentals section.

Preparation of polymers (previously disclosed in DK application PA200600337).

The polymers can in principle be prepared following principles known to the person skilled in the art.

In principle, polymer where B is not a residue A (diblock-type polymers) can be prepared as follows:

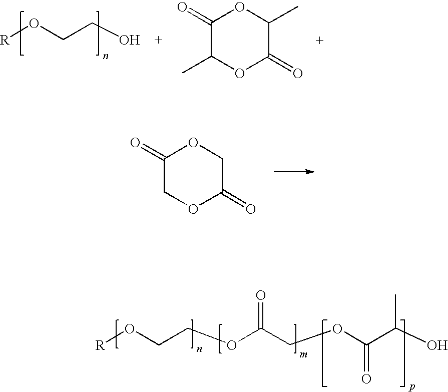

In principle, polymer where B is a residue A (triblock-type polymers) can be prepared as follows:

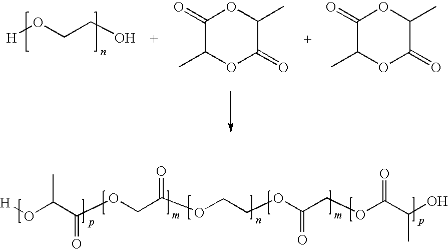

Unless special conditions are applied, the distribution of lactide units and glycolide units will be randomly distributed or tapered within each poly(lactide-co-glycolide) residue.

Preferably the ratio of glycolide units and lactide units present in the polymer used in scaffold is between an upper limit of about 80:20, and a lower limit of about 10:90, and a more preferable range of about 60:40 to 40:60.

Preferably the upper limit of PEG-content is at most about 20 molar %, such as at most about 15 molar %, such as between 1-15 molar %, preferably between 4-9 molar %, such as about 6 molar %.

The synthesis of the polymers according to the invention is further illustrated in the Experimentals section.

Further Aspects of the Scaffold (as disclosed in DK application PA200600337, but which may also be applied to other scaffolds)

The scaffold may, e.g. be a biodegradable, porous material comprising a polymer as defined herein, wherein the porosity is at least 50%, such as in the range of 50-99%.

The high degree of porosity can be obtained by freeze-drying.

The void space of the material of the polymer may be unoccupied so as to allow or even facilitate cell adhesion and/or in-growth for regeneration of tissue. In one embodiment, however, the pores of the material are at least partly occupied by a component from the extracellular matrix. Such components may facilitate the cell adhesion and/or in-growth for regeneration of tissue. Examples of components from the extracellular matrix are chondroitin sulfate, hyaluronan, hyaluronic acid, heparin sulfate, heparan sulfate, dermatan sulfate, growth factors, fibrin, fibronectin, elastin, collagen, gelatin, and aggrecan.

The scaffold may also contain the conversion component thrombin either alone or in combination with one of the above mentioned.

The components from the extracellular matrix could be added either as particles, which are heterogeneously dispersed, or as a surface coating. The concentration of the components from the extracellular matrix relative to the synthetic polymer is typically in the range of 0.5-15% (w/w), preferably below 10% (w/w). Moreover, the concentration of the components of the extracellular matrix is preferably at the most 0.3% (w/v), e.g. at the most 0.2 (w/v), relative to the volume of the material.

The porous materials may be prepared according to known techniques, e.g. as disclosed in Antonlos G. Mikos, Amy J. Thorsen, Lisa A Cherwonka, Yuan Bao & Robert Langer. Preparation and characterization of poly(L-lactide) foams foams. Polymer 35, 1068-1077 (1994). One very useful technique for the preparation of the porous materials is, however, freeze-drying.

In one embodiment, the scaffold may be prepared by the following method which is previously disdosed in DK application PA200600337. The method is particularly suited prepare scaffolds from PLGA and MPEG-PLGA polymers.

(a) dissolving a polymer as defined herein in a non-aqueous solvent so as to obtain a polymer solution;

(b) freezing the solution obtained in step (a) so as to obtain a frozen polymer solution; and

(c) freeze-drying the frozen polymer solution obtained in step (b) so as to obtain the biodegradable, porous material.

The non-aqueous solvent used in the method should with respect to melting point be selected so that it can be suitable frozen. An illustrative examples hereof arels dioxane (mp. 12° C.) and dimethylcarbonate (mp. 4° C.).

In one variant, the polymer solution, after step (a) is poured or cast into a suitable mould. In this way, it is possible to obtain a three-dimensional shape of the material specifically designed for the particular application.

Particles of components from the extracellular matrix may be dispersed in the solution obtained in step (a) before the solution (dispersion) is frozen at defined in step (b).

The components from the extracellular matrix may, for instance, be dissolved in a suitable solvent and then added to the solution obtained in step (a). By mixing with the solvent of step (a), i.e. a solvent for the polymer defined herein, the components from the extracellular matrix will most likely precipitate so as to form a dispersion.

In one aspect, the biodegradable, porous material obtained in step (c), in a subsequent step, is immersed in a solution of glucosaminoglycan (e.g. hyaluronan) and subsequently freeze-dried again.

In some alternative embodiments, the material are present in the form of a fibre or a fibrous structure prepared from the polymer defined herein, possibly in combination with components from the extracellular matrix. Fibres or fibrous materials may be prepared by techniques known to the person skilled in the art, e.g. by melt spinning, electrospinning, extrusion, etc.

### EXAMPLES

### Example 1

A hydrophilic scaffold containing human cells embedded in a hydrogel according to the invention was prepared in the following manner.

The MPEG-PLGA scaffold (prepared according to the examples from DK application PA2006 00337 which are provided for reference below) was cut out with a sterile scalpel to a circular shape of 10 mm in diameter.

In regards to the species of thrombin used in the example in this context, was bovine thrombin used as thrombin “In general” for the examples, but the resulting gelation would be the same with human thrombin. Although, as discussed herein, the use of bovine thrombin should be avoided in a composition or kit according to the present invention, the thrombin used in the examples is merely to demonstrate the gelating effect thrombin with no respect to the origin of the thrombin. This thrombin was prepared by dissolving the thrombin in sterile H₂O containing 0.1% BSA.

In regards to the species of fibrinogen used in the example in this context, was bovine fibrinogen used as fibrinogen “in general” for the examples, but the resulting gelation would be the same with human fibrinogen. Although, as discussed herein, the use of bovine fibrinogen should be avoided in a composition or kit according to the present invention, the fibrinogen used in the examples is merely to demonstrate the gelating effect fibrinogen with no respect to the origin of the fibrinogen, only to its purity which is over 90% pure fibrinogen. This fibrinogen was prepared by dissolving the fibrinogen in DMEM/F12 (Life Technologies) containing gentamicin sulphate (Invitrogen) at a final concentration of 59 μg/ml medium and fungizone (Invitrogen) at a final concentration of 2.4 μg/ml at 37° C.

6 μl of the above thrombin solution, kept at 4° C. was added to 494 μL sterile 40 mM CaCl₂. This activated thrombin solution (500 μL) was drawn into a tuberculin (1 ml) syringe and kept at 4° C. until use.

500 μl of the above fibrinogen solution, kept at a temperature 37° C. (normally in a range between 4° C. and 37° C.) was drawn into a tuberculin (1 ml) syringe, any number of chondrocytes could be added to provide a final number of cells into a cartilage defects such as for Instance 0.1×10⁴ to 10×10⁶ human chondrocytes/cm² added and the cell containing solution was kept at a temperature range between 4°-37° C. until use and with an upper acceptable maximum temperature of 38° C.

The MPEG-PLGA scaffold was placed in a sterile Petri-dish (Nunc) ready to be loaded with the hydrogel composed of chondrocytes and fibrinogen/thrombin. The two syringes were combined with a Y-connecter. The two pistons of the tuberculin syringes were activated using a steady pressure. 250 μL were loaded onto the MPEG-PLGA scaffold placed in the Petri-dish. After 30-60 sec. the hydrogel were absorbed due to the hydrophilic nature of the MPEG-PLGA scaffold. After absorption a coagulation process is initiated within the MPEG-PLGA scaffold, thus retaining the chondrocytes within the scaffold.

Following coagulation 25 ml DMEM/F12 containing 20% fetal calf serum, gentamicin, 59 μg/ml and fungizone, 2.5 μg/ml as well as P-ascorbate in order to study the behavior of the chondrocytes over a 3 week period within the scaffold. The scaffold were placed in a 5% CO₂ Incubator at 37° C.

### Example 2

A hydrophilic scaffold containing human cells embedded in a hydrogel according to the invention was prepared in the following manner. The Trufit® from Osteobiologics, Inc., for pre-clinical studies called PolyGraft® BGS, Lot # X41053, to be used for pre-clinlcal studies only, was measuring 5.3×3 mm, and packed in a sterile package consisting of aluminum foil.

In regards to the species of thrombin used in this example and in this context, similar to the above described Example 1, was bovine thrombin used as thrombin “in general” for the examples, but the resulting gelation would be the same with human thrombin. Although, as discussed herein, the use of bovine thrombin should be avoided in a composition or kit according to the present invention, the thrombin used in the examples is merely to demonstrate the gelating effect thrombin with no respect to the origin of the thrombin. This thrombin was prepared by dissolving the thrombin In sterile H₂O containing 0.1% BSA.

In regards to the species of fibrinogen used in the example in this context, was bovine fibrinogen used as fibrinogen “In general” for the examples, but the resulting gelation would be the same with human fibrinogen. Although, as discussed herein, the use of bovine fibrinogen should be avoided in a composition or kit according to the present invention, the fibrinogen used in the examples is merely to demonstrate the gelating effect fibrinogen with no respect to the origin of the fibrinogen, only to its purity which is over 90% pure fibrinogen. This fibrinogen was prepared by dissolving the fibrinogen in DMEM/F12 (Life Technologies) containing gentamicin sulphate (Invitrogen) at a final concentration of 59 μg/ml medium and fungizone (Invitrogen) at a final concentration of 2.4 μg/ml at 37° C. 6 μl of the above thrombin solution, kept at 4° C. was added to 494 μL sterile 40 mM CaCl₂. This activated thrombin solution (500 μL) was drawn into a tuberculin (1 ml) syringe and kept at 4° C. until use.

500 μl of the above fibrinogen solution, kept at a temperature 37° C. (normally in a range between 4° C. and 37° C.) was drawn into a tuberculin (1 ml) syringe, any number of chondrocytes could be added to provide a final number of cells into a cartilage defects such as for instance 0.1 to 10×10⁵ human chondrocytes/cm² added and the cell containing solution was kept at a temperature range between 4°-37° C. until use and with an upper maximum temperature of 38° C.

The Trufit® was placed in a sterile Petri-dish (Nunc) ready to be loaded with the hydrogel composed of chondrocytes and fibrinogen/thrombin. The two syringes were combined with a Y-connecter. The two pistons of the tuberculin syringes were activated using a steady pressure. 250 μL were loaded onto the Trufit® placed in the Petri-dish. After 30-60 sec. the hydrogel were absorbed due to the hydrophilic nature of the Trufit®. After absorption a coagulation process is initiated within the Trufit®, thus retaining the chondrocytes within the Trufit®.

Following coagulation 25 ml DMEM/F12 containing 20% fetal calf serum, gentamicin, 59 μg/ml and fungizone, 2.5 μg/ml as well as P-ascorbate in order to study the behavior of the chondrocytes over a 3 week period within the Trufit®. The Trufit® were placed in a 5% CO₂ incubator at 37° C.

### Example 3

Human articular chondrocytes (hACs) were cultured as a monolayer cell culture and then released from the cell culture flask using trypsin—EDTA.

0.5×10⁶ cells were combined with the hydrogel composed of chondrocytes and fibrinogen/thrombin, and subsequently applied to the special Trufit (called Poly-Graft Top Phase from Osteobiologics, San Antonio, Tex.) in a Petri dish. After 5 minutes the “Hydrogel-PolyGraft System was placed in a 24 well plate and growth medium was applied to the wells. 6 (six) samples were cultured at 37° C. In a CO₂ incubator; growth medium were changed around twice a week. 2 (two) samples were kept for 14 days to get a “14 day” time point analysis, 2 (two) for 2 months analysis and 2 for 4 months analysis.

After each time point the following analysis were conducted:
Cryo-section followed by histology (Toluldine Blue Staining and Safranin O staining) and immunocytochemistry (monoclonal antibodies against Aggrecan and Collagen type II)
RNA purification for subsequent RT-PCR analysis. The expression of Collagen type II and the chondrogenic transcription factor Sox9 were analyzed. For these experiments the controls were hACs cultured as a monolayer in the same growth medium used for the culturing in the Hydrogel-Polygraft Systems.
Migration-assay. After each time point each Hydrogel-PolyGraft system was processed into explants and the migration of hACs were observed under light microscopy (LM).

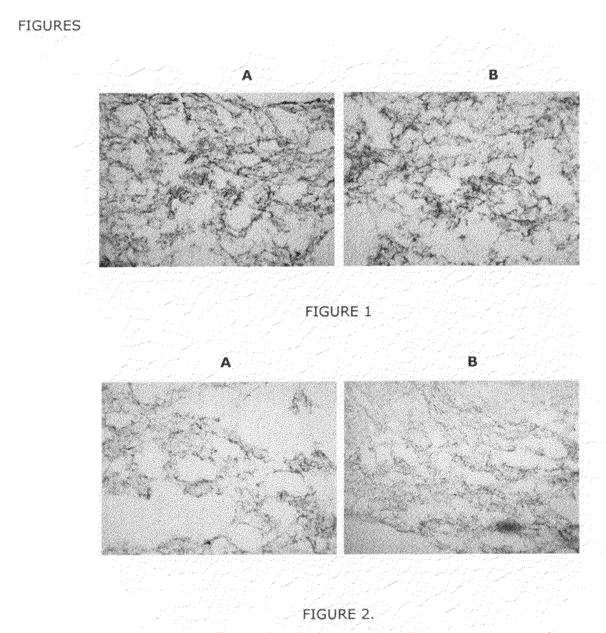

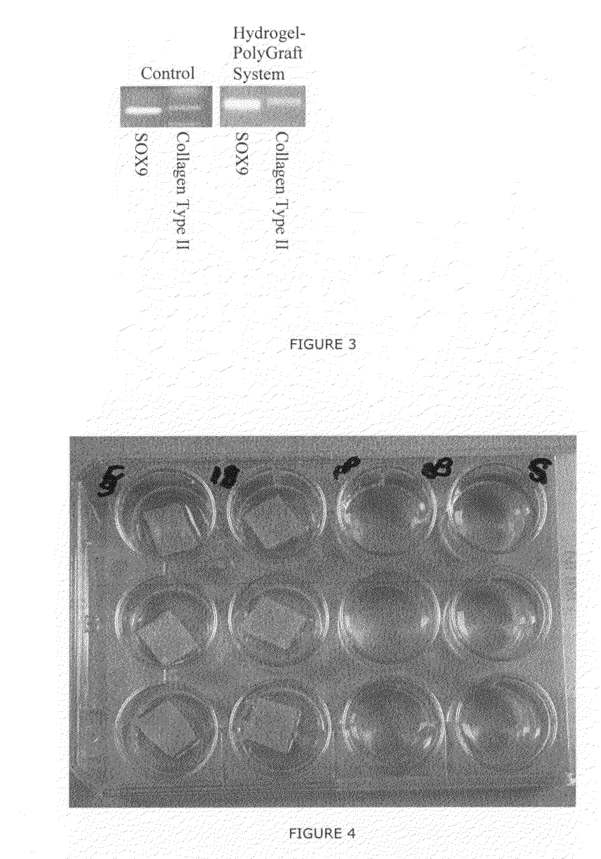

The results shown below, are “14 days time point”. Histology is shown in FIG. 1, Immunohisto-chemistry is shown in FIG. 2, and Gene Expression is shown In FIG. 3.

### Migration Assay

A nice and fast cell migration was observed post-processing. This migration rate is comparable to normal migration in normal articular cartilage tissue.

Although these data were already found after the 14 days time point hACs cultured within this Hydrogel-Polygraft System were able to synthesize small amounts of cartilage matrix enriched in proteoglycans as demonstrated by Toluldine Blue—and Safranin O staining. By Immuno-histochemistry it was furthermore demonstrated that hACs cultured in this system synthesized both collagen type II and aggrecan; two well known markers for articular hyaline cartilage.

In addition, gene expression of collagen type II and the chondrogenic transcription marker SOX9 were demonstrated by RT-PCR. The expression of collagen type II confirmed the expression on the protein—level as demonstrated by Immunohistochemistry.

Furthermore, the gene expression analysis demonstrated that a much lower mRNA level of these two markers were present in the chondrocyte monolayer control cultures, when compared to the relatively high expression of mRNA level of the markers in the Hydrogel-PolyGraft (PolyGraft itself was also called PolyGraft Top Phase).

We expect that the signals measured by the above methods will increase strongly with increasing culture—time.

### Example 4

In another experiment, we used MPEG-PLGA scaffolds as a SCAS system. The scaffolds were again produced with various concentrations of 2 different hyaluronic acid (HA and NZHA) ranging from 1% to 10%, or coated with these or produced with CS (chodroritin sulfate) Each scaffold was cut aseptically into triplicates of 1×1 cm and placed in triplicates in sterile 12 well flat bottom trays. Each triplicate was given code numbers for the particular scaffold. The Code numbers contain the following coating of the MPEG-PLGA

The following Code numbers for the membranes used were as follows:

### HA=Hyaluronic Acid

### MPEG-PLGA=(Methoxypolyethyleneglycol-block-co-poly(lactide-co-glycolide).

In this study the composition of MPEG-PLGA is the same as described herein.

The scaffold is produced by freeze drying. The scaffolds are sheets with thickness of 1-3 mm and porosity around 90%.

### Code 1: PLGA 4%, 1% Bulk HA

MPEG-PLGA (Mw 2.000-30.000 Da; the PLA/PGA ratio is 50:50) “casted” from a 4 w/w % dioxane solution. The Scaffold contains 1 w/w % HA (Mw above 1 mill Da) as particles. The scaffold in produced by freeze drying. The scaffold is a sheet with thickness of 1-3 mm and porosity around 90%.

### Code 2: PLGA 4%, Coated 1.5% Bulk HA,

MPEG-PLGA Scaffold casted from a 4 w/w % dioxin solution. The scaffold is freezed dried. Then it is coated with 1.5 w/w % HA solution and dried. (Mw HA>1 mill)

### Code 3: PLGA 4%, 2% Bulk HA,

See No 1. The only different is that this contains 2 w/w % HA as particles.

### Code 4: PLGA 4%, 5% Bulk HA,

See No 1. The only different is that this contains 5 w/w % HA as particles.

### Code 5: PLGA 4%, 10% Bulk HA,

See No 1. The only different is that this contains 10 w/w % HA as particles.

### Code 6: PLGA 4%, 1% NZHA,

See No 1. The only different is that this contains 1 w/w % HA (Mw around 700.000 Da) as particles.

### Code 7: PLGA 4%, 2% NZHA,

See No 1. The only different is that this contains 2 w/w % HA (Mw around 700.000 Da) as particles.

### Code 8: PLGA 4%, 5% NZHA,

See No 1. The only different is that this contains 5 w/w % HA (Mw around 700.000 Da) as particles.

### Code 9: PLGA 4%, 10% NZHA,

See No 1. The only different is that this contains 10 w/w % HA (Mw around 700.000 Da) as particles.

### Code 10: PLGA 4%,

Scaffold made from MPEG-PLGA (Mw 2.000-30.000 Da; the PLA/PGA ratio is 50:50) “casted” from a 4 w/w % dioxane solution with no additives.

A gelatin based scaffold may be prepared, such as gelatine 1%, 1-10% bulk HA, This is routinely made using a 1 w/w % solution of gelatine containing 1 w/w % HA (Mw 1 mill). The scaffold is cross linked with EDC.

A human chondrocyte culture obtained using the explant method (described in PCT/DK02/00065 patent application published under WO 02/061052) was expanded up to 16×10⁶ human chondrocytes in cell culture flasks using DMEM/F12 containing 16% foetal calf serum and gentamicin and fungizone. The chondrocytes were re-suspended to a concentration of 4×10⁶ cells per ml (2×10⁵ cells per 50 ul) In serum-free DMEM/F12 containing 50 mg/ml of bovine fibrinogen F 8630, product description CAS Number: 9001-32-5 (the fibrinogen was dissolved at 37° C. in the medium within 5 to 60 minutes prior to use). An amount of 50 ul of the cell/fibrinogen solution was added to each scaffold (except for two rows of wells in the plate carrying the Codes 1 and 2 as well as the upper well of Code 3, which by accident received 197 ul cell/fibrinogen; the excess cell/fibrinogen solution was immediately removed).

The cell/fibrinogen solution was allowed to be soaked into the various scaffolds tested. Some scaffolds showed readily suction within seconds, when the solution was applied. Other scaffolds showed only minute degree of suction, and the 50 ul was forming a drop on top of these scaffolds, even after 2-3 minutes.

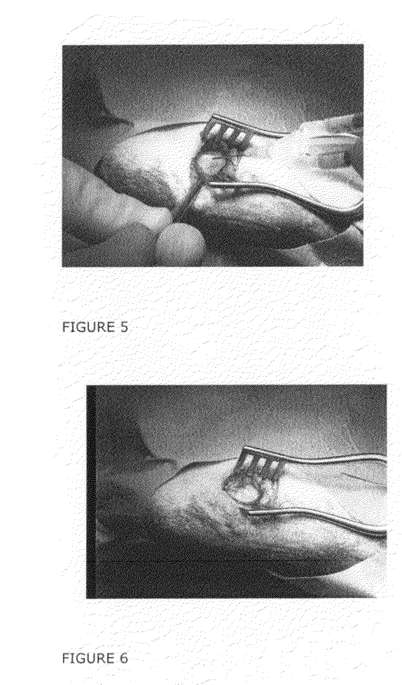

As the other part of the constituent 50 ul of the thrombin solution was added to each of the 1×1 cm scaffolds. The thrombin was from bovine plasma, Product Number T6634, Production Description 9002-04-4 and was prepared at a concentration of 100 units/ml (corresponding to 0.1 unit per μl). An amount of 5 μl of thrombin (corresponding to 0.1 unit×5 μl)=0.5 units was added to 495 μl of a 40 mM CaCl₂ solution. Of this diluted solution 50 ul of the thrombin was added to each scaffold. The following results were observed during the experiments as described under each of the FIGS. 4 through 7.

Fifty (50) μl, consisting of two hundred thousand (200,000) human chondrocytes/50 μl DMEM/F12 culture medium, without any serum, except 50 mg/ml bovine fibrinogen previously dissolved in the medium, were placed on top of each scaffold. Fifty (50) μl of bovine thrombin pre-diluted in 40 Mm Calcium chloride solution at 4° C. was added. The set up consisted of triplicates of each type of scaffold. In this setup the Code numbers of the various scaffolds, and the results of the application are shown in Table 1. Clotting was allowed for 5 minutes at room temperature and the absorption time of the total of 100 μl applied on the scaffolds was estimated. After 5 minutes two (2) ml of DMEM/F12 culture medium, containing 16% vol/vol foetal calf serum and antibiotics such as gentamicin and fungizone, was then added to each well, the form and the adherence of the individual scaffolds were noted. The plates were incubated at 37° C. in a CO₂ incubator. After 3 days of incubation the medium was removed and new medium was added. These so called “SCAS” systems or membranes, Code 1 through 2 as well as the top well Code 3 got by mistake initially 192 μl cell/fibrinogen mix. The excess that was not soaked into the membrane in these wells, and the surplus were immediately removed as thoroughly as possible. An amount of 50 ul of cell/fibrinogen mix was added to the rest of the membranes in the rest of the wells in the entire experiment. Fifty (50) ul of the thrombin solution, diluted as described previously, was added to each well. After adding the thrombin, some of the total solution was not soaked into wells coded 1 and 2 as well as the upper well of Code 3, but appeared to have dispersed into the well (which also appeared to be adherent to the bottom of the well).

An example of the testing of the SCAS principle (cells/fibrin/scaffold in wells) is shown In FIG. 4.

TABLE 1
Application of human fibrin/chondrocytes on
variation of “ColoPlast” scaffolds

| | | | | Migration |
|---|---|---|---|---|
| | Time of | Adherence of | Form of the | of cells in |
| | Soaking of | the scaffold to | scaffold on the | well, |
| Scaffold | cell/fibrin to | the bottom of | bottom of the | outside the |
| Code # | the scaffold | the tray well | tray well | scaffold |
| Code 1 | <1 min. | +++ | Flat, no folding | +++ |
| Code 2 | <1 min. | +++ | Flat, no folding | +++ |
| Code 3 | <1 min. | +++ | Flat, no folding | +++ |
| Code 4 | <1 min. | +++ | Flat, no folding | +++ |
| Code 5 | <1 min. | +++ | Flat, no folding | +++ |
| Code 6 | >2 min. | (+)+ | Flat, no folding | +++ |
| Code 7 | 2 min. | ++ | Flat, no folding | +++ |
| Code 8 | 2 min. | ++ | Flat, no folding | +++ |
| Code 9 | 2 min. | ++ | Flat, no folding | (+)+ |
| Code 10 | <1 min. | +++ | Flat, no folding | +++ |

### Example 5

The SCAS system was tested in the femoral condyle of 10 adult goats In the operation theater at the Research Center at Foulum. Fibrin/“autologous” chondrocyte were mixed and applied to the scaffold.

The SCAS system was compared to 3 groups; 1. Empty defect (control), 2. chondrocytes mixed with fibrin gel (FIB50) and 3. microfracture (An already established method to treat articular cartilage defects).

A 6 mm circular defect was created in both medial femoral condyles in the adult goats used for the study. Cartilage tissue was harvested for chondrocyte culture. At secondary open surgery the defects were randomized to the four treatment groups (10 knee joints in each group).

The treatment with the SCAS system consisted of the following steps.

The surgeon was provided with the following 3 vials 2 hours before surgery; (1) chondrocytes (1×10⁶ cells/100 μl) suspended In DMEM/F12 containing 16% foetal calf serum and antibiotics, (2) fibrinogen solution (100 mg/ml DMEM/F12+antibiotics) and (3) thrombin solution (100 U/ml CaCl₂). Furthermore the surgeon was provided with MPEG-PLGA scaffolds (1 cm×1 cm).

Just before surgery the chondrocyte suspension was aseptically mixed with the fibrinogen solution (1:1 v/v) and the chondrocyte/fibrinogen solution was drawn into a tuberculin syringe (1 ml). The thrombin solution was drawn into another tuberculin syringe (1 ml) and the two syringes were combined; now forming a double-syringe.

Half of the volume in the two chambers of the double syringe was then applied to the bottom of the defect in the knee joint of the goat (FIG. 5). The MPEG-PLGA scaffold was then placed into the scaffold and finally the remaining solution in the two chambers of the double-syringe was applied to the MPEG-PLGA scaffold. Subsequently the knee-joint was left untouched for 5 min. in order to allow the MPEG-PLGA scaffold to absorb the chondrocytes/hydrogel. After 5 min. the joint were closed.

The animals were followed for 4 month. Analyses: ICRS macroscopic scoring (0-12). Mechanical test was performed to assess stiffness of regeneration tissue. Histological analyses was performed by O, Driscoll and Pinada cartilage scores and percentage filling of the defects were determined.

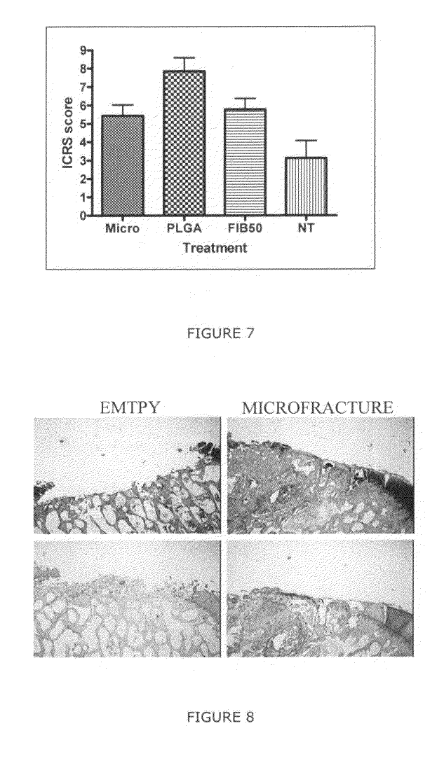

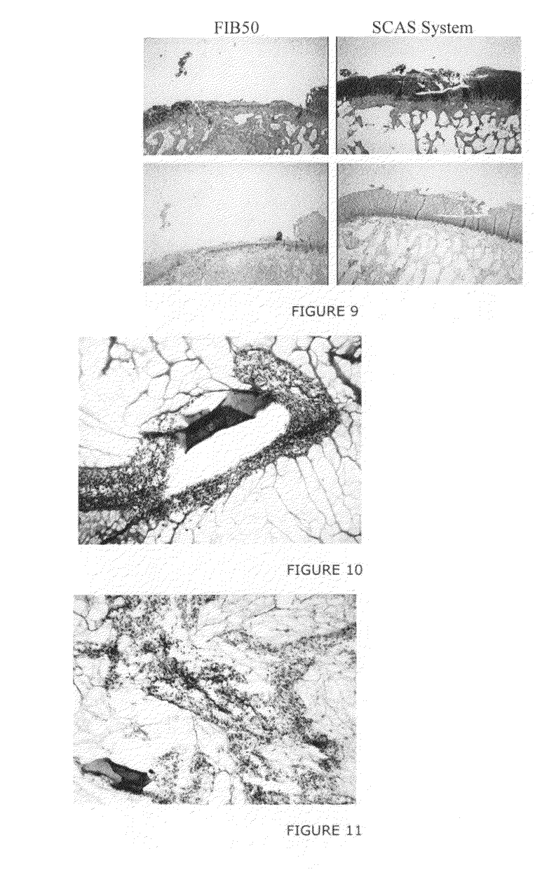

The ICRS macroscopic scores and histology appearance demonstrated highly significant difference between groups (FIG. 7 to 9). The cartilage regeneration with the SCAS system demonstrated high defect fill and a tissue characteristic close to hyaline cartilage whereas no hyaline regeneration tissue was seen in the empty defects. Mechanical testing demonstrated no difference between treatment groups.

The SCAS System demonstrated an extensive cartilage regenerative response with good phenotypic characteristic. As expected no regeneration was seen in the empty defects. The method appeared to be an extremely good technique for cartilage tissue engineering in vivo, creating hyaline-like articular cartilage in the defects.

### Example 6

In order to determine if the MPEG-PLGA loaded with a hydrogel composed of fibrinogen/thrombin, would allow migration of chondrocytes from an intact cartilage tissue into the MPEG-PLGA/hydrogel scaffold the following experiment was done.

Small cartilage explants (3-5) were generated from cartilage biopsies, obtained from normal articular cartilage and placed within MPEG-PLGA scaffolds (1 cm×1 cm). The scaffolds containing the cartilage explants were placed in wells (12 wells plate). 50 μl serum-free DMEM/F12 containing 50 mg/ml of bovine fibrinogen F 8630, product description CAS Number: 9001-32-5 (the fibrinogen was dissolved at 37° C. In the medium within 5 to 60 minutes prior to use) was applied to the MPEG-PLGA scaffolds and after absorption into the scaffolds 50 ul of the thrombin solution was added to scaffolds. The thrombin was from bovine plasma, Product Number T6634, Production Description 9002-04-4 and was prepared at a concentration of 100 units/ml (corresponding to 0.1 unit per μl). An amount of 5 μl of thrombin (corresponding to 0.1 unit×5 μl)=0.5 units was added to 495 μl of a 40 mM CaCl₂ solution. Of this diluted solution 50 ul of the thrombin was added to each scaffold.

After 5 min. 3 ml growth medium was added to the well and the scaffold was cultured for 3 weeks, with a medium change every 3-4 days.

The scaffolds were finally snap-frozen and subsequently cryo-sectioning was performed. Sections were fixed and stained with toluidine blue.

The following results were observed during the experiments as described under each of the FIGS. 10 and 11.

This experiment demonstrates that the MPEG-PLGA/Hydrogel scaffold (SCAS) allows chondrocytes from cartilage tissue to migrate into the scaffold structure. This is important as, migration of chondrocytes from the cartilage surrounding a defect in the human knee joint into the scaffold, is essential for an optimal hyaline-like articular cartilage regeneration-response.

### Example 7

### (From DK Application PA2006 00337)—Polymer Biodegradation Test Biodegradability of the Porous Material can be Determined as Follows

Approx. 1 gram of a porous material is fully immersed in a medium (10% foetal calf serum in DMEM (Dulbecco's modified Eagle's medium)) and is stored at 37° C. for a period of 28 days. The medium is changed twice a week, i.e. on days 3, 7, 10, 14, 17, 21, and 24. On day 28, the porous material is analysed by GCP. The biodegradation is measured as the number/weight average molecular weight relative to the initial value.

A porous MPEG-PLGA (2-30 kDa, L:G 50:50) was tested, and the biodegradation was determined as approx. 0.5 (final M_{n/w} value relative to Initial value)

### Summary

As an attempt to make PLGA more hydrophilic, MPEG or PEG was copolymerised with PLGA to give copolymers with a low MPEG/PEG-content (<20% MPEG/PEG). When these polymers were tested and compared to plain PLGA, the initial adhesions of cells to MPEG-PLGA and PLGA-PEG-PLGA were superior to plain PLGA and the morphology and attachment of the cells were better.

This is surprising, since PEG-containing polymers are known from the literature to resist the adhesion of proteins and cells. The key to the improved performance of our polymers seems to be that the PEG-content in the polymer is kept low (at the most 20 mol-%, preferably at the most 14 mol-%) as polymers with high PEG-content gave poor adhesion and morphology in the biological tests.

PLA have long degradation-times compared to PLGA, and our experiments show that a higher lactide content in the PLGA-part of polyether-PLGA give a slower adhesion of cells.

Known synthetic biodegradable polymers are typically hydrophobic materials with sluggish initial cell adhesion in a biological environment. We attempted to modify the hydrophilicity of PLGA by synthesizing an MPEG-PLGA block copolymer. Our first polymer was a 1.9-30 kDa MPEG-PLGA with an G:L-ratio of 50:50 (mol). These are made into thin porous sheets by freeze-drying. In a biological assay both the initial and long-term cell adhesion was excellent, and the performance was superior to the unmodified PLGA. This is surprising, since the literature describes that incorporation of PEG into polymers make them resistant to the adhesion of cells and proteins. The key to our success seems to be that we have a low PEG-content (6%). When the PEG content is higher (MPEG-PLGA 5-30 kDa, 14% PEG) we see a reduced cell adhesion, both initial and longer term when compared to the low-PEG materials and plain PLGA.

### Example 8

### (From DK application PA2006 00337)—Purification of polymer

The polymer is dissolved in a suitable solvent (e.g. dioxane, tetrahydrofuran, chloroform, acetone), and precipitated with stirring in a non-solvent (e.g. water, methanol, ethanol, 1-propanol or 2-propanol) at a temperature of −40 to 40° C. The polymer is left to settle, solvent discarded and the polymer is dried in a vacuum oven at 40-120° C./overnight.

The polymers are analyzed with NMR-spectroscopy and GPC to confirm structure, molecular weight and purity.

### Examples of the Synthesis of Various MPEG-PLGA Polymers

| | | | | | | 4% | |
|---|---|---|---|---|---|---|---|
| | G/L- | glycolide | DL-lactide | | initiator | Sn(Oct)2 | Dioxane |
| Polymer | ratio | (g) | (g) | Initiator | (g) | (μL) | (g) |
| 750- | 50:50 | 2.12 | 2.64 | MPEG 750 Da | 0.238 | 129 | 5 |
| 15000 | | | | | | | |
| 50L | | | | | | | |
| 1100- | 50:50 | 2.11 | 2.63 | MPEG 1100 Da | 0.261 | 98 | 5 |
| 20000 | | | | | | | |
| 50L | | | | | | | |
| 1900- | 50:50 | 2.10 | 2.60 | MPEG 1900 Da | 0.298 | 65 | 5 |
| 30000 | | | | | | | |
| 50L | | | | | | | |
| 1900- | 20:80 | 0.79 | 3.91 | MEPEG 1900 Da | 0.298 | 65 | 5 |
| 30000 | | | | | | | |
| 80L | | | | | | | |
| 5000- | 50:50 | 1.86 | 2.31 | MPEG 5000 Da | 0.833 | 68 | 5 |
| 30000 | | | | | | | |
| 50L | | | | | | | |
| 15000- | 50:50 | 2.10 | 2.60 | PEG 1900 Da | 0.298 | 65 | 5 |
| 1900- | | | | | | | |
| 15000 | | | | | | | |
| 50L | | | | | | | |
| 30000- | 50:50 | 2.06 | 2.56 | PEG 5000 Da | 0.385 | 31 | 5 |
| 5000- | | | | | | | |
| 30000 | | | | | | | |
| 50L | | | | | | | |

### Process for Making Scaffolds

Polymer (e.g. 1.9-30 kDa) is dissolved in a suitable solvent (e.g. dioxane) to a concentration of 0.5-10% (w/v). The solution is poured in a mold, frozen and freeze-dried to at porous sheet. Component from the extracelluar matrix may be incorporated either by dispersing such components in the solvent or by subsequently treating the porous sheet with a dispersion/solution of components from the extracellular matrix.

### Testing of Scaffolds

Biocompatibility studies of the different scaffolds of MPEG-PLGA and PLGA were performed by seeding primary fibroblast at a concentration of 2.5×10⁴ cells/cm² on the surface of the scaffolds. Evaluation of the cells attachment, viability and growth were preformed at day 1, 3 and 7 by staining the cells using neutral red followed by evaluation using an Lelca DMIRE2 inverted microscope fitted with a Evolution MP cooled color camera (Media Cybernetics) and digital images were taken using image Pro Plus 5.1 software (Media Cybernetics).

Studies comparing the biocompatibility of freeze dried scaffolds of PLGA showed generally adhering of cells with fine morphology but very low initial amount of cells. Comparing these scaffolds with MPEG-PLGA 2-30 kDa, we see a better biocompatibility of the MPEG-PLGA scaffold because of a higher amount of cells are adhering to this scaffold due to a better wetting ability.

Cells are growing with a fine morphology and good adherence to MPEG-PLGA 1.9-30 kDa from the start of the test and an increase in amount of cells are seen from day 1 to day 7. Increasing the size of the MPEG part of the MPEG-PLGA to 5-30 kDa gives solely rounded cells with little or no adherence to the surface of the scaffolds giving a pronounced decreased biocompatibility and which is worsened from day 1 to day 7.

If MPEG-PLGA 2-15 kDa is tested and compared with MPEG-PLGA 2-20 kDa and MPEG-PLGA 2-30 kDa, we see an increasing attachment and viability of the fibroblasts when the size of the PLGA part was increased. This means that the 2-30 kDa had the best biocompatibility. Increasing the size from 2-15 kDa to 2-20 kDa gives the largest positive effect on the biocompatibility compared with the step from 2-20 kDa to 2-30 kDa.

Increasing the lactide content in MPEG-PLGA 2-20 kDa from 60% to 80 mol % gives decreased attachment and viability of the fibroblasts. This effect is more pronounced when MPEG-PLGA 2-15 kDa 60% lactide are compared with MPEG-PLGA 2-15 with 80% lactide.

### Summary of the Biocompatibility Tests:

| Scaffold | | LA | % MPEG/ | Attachment | | | Viability | | |
|---|---|---|---|---|---|---|---|---|---|
| type | size | %²⁾ | PEG | Day 1 | Day 3 | Day 7 | Day 1 | Day 3 | Day 7 |
| PLGA¹⁾ | — | 50 | 0 | + | ++ | ++++ | +++ | +++ | ++++ |
| MPEG- | 2-15 | 50 | 12 | + | + | + | ++ | ++ | ++ |
| PLGA | 2-15 | 60 | 12 | + | + | + | ++ | ++ | + |
| | 2-15 | 80 | 12 | + | + | + | + | + | + |
| | 2-20 | 50 | 9 | ++ | +++ | +++ | ++ | +++ | +++ |
| | 2-20 | 60 | 9 | ++ | ++ | ++ | ++ | ++ | ++ |
| | 2-20 | 80 | 9 | + | + | + | + | + | + |
| | 2-30 | 50 | 6 | +++ | +++++ | +++++ | ++++ | +++++ | +++++ |
| | 2-30 | 60 | 6 | +++ | ++++ | +++++ | ++++ | +++++ | +++++ |
| | 2-30 | 80 | 6 | +++ | ++++ | ++++ | +++ | ++++ | ++++ |
| | 5-25 | 50 | 17 | + | + | + | + | + | + |
| | 5-30 | 50 | 14 | ++ | + | + | ++ | + | + |
| | 5-79 | 50 | 6 | +++ | ++++ | +++++ | ++++ | +++++ | +++++ |
| PLGA- | 13-6-13 | 50 | 20 | + | + | + | + | + | + |
| PEG- | 23-3-23 | 50 | 6 | +++ | +++++ | +++++ | ++++ | +++++ | +++++ |
| PLGA | 47-6-47 | 50 | 6 | ++ | +++ | ++++ | +++ | +++ | +++++ |

¹⁾Alkermes MEDISORB PLGA 5050DL high I.V.
²⁾LA as mol % of the PLGA part of the polymer

The results are graded subjective from + to +++++ with + meaning low attachment and low viability while +++++ are excellent attachment and viability.

Human keratinocytes can be cultured in vitro on fibroblast populated MPEG-PLGA scaffolds to form a multilayered and differentiated reconstituted epidermis (see FIG. 1). The reconstituted epidermis shows morphological features resembling normal epidermis in vivo.

On histological specimens, we find clear evidence of basal cell layers (stratum basale) and ultimately overlying stratum corneum with intervening layers resembling, however, immature and slightly hyperproliferative, spinous and granular layers. Lack of final maturation should be ascribed to the chosen in vitro model rather than the scaffold material.

### Example 9

### Cartilage Regeneration with Chondrocytes in (MPEG-PLGA) Polylactate Scaffold. an In Vivo Study in Goats

Recently porous scaffolds have been introduced for clinical cartilage tissue engineering. Numerous scaffold materials exist and the optimal scaffold needs to be identified. The present study alms to Investigate the cartilage regenerative response of a MPEG-PLGA porous scaffold combined with chondrocyte suspension in a goat femoral condyle full thickness cartilage defect model.

### Methods

10 adult goats were used for the study and the study conducted at the Research Center at Foulum, Denmark. A 6 mm circular defect was created in both medial femoral condyles. Cartilage tissue was harvested for chondrocyte culture. At secondary open surgery the defects were randomized to the following two treatment groups. 1. Empty defect (control) 2. Fibrin/chondrocyte solution in a MPEG-PLGA freezed dried porous scaffold. Animals were followed for 4 month. Analyses: ICRS macroscopic scoring (0-12). Mechanical test was performed to assess stiffness of regeneration tissue. Histological analyses was performed by O, Driscoll and Pinada cartilage scores and percentage filling of the defects.

### Results

The ICRS and histology scores demonstrated highly significant difference between groups. The cartilage regeneration is MPEG-PLGA/Cell group demonstrated high defect fill and a tissue characteristic close to hyaline cartilage whereas no regeneration tissue was seen in the empty defects. Mechanical testing demonstrated no difference between treatment groups.

### Conclusion

The MPEG-PLGA/cell construct demonstrates an extensive cartilage regenerative response with good phenotypic characteristic. As expected no regeneration was seen in the empty defects. A porous MPEG-PLGA scaffold in combination with cultures chondrocytes seem to be a good technique for cartilage tissue engineering in vivo.

### Example 10

### Cartilage Regeneration with Chondrocytes in Fibrinogen Gel Scaffold and Microfracture. an In Vivo Study in Goats

### Introduction

The present clinical focus of cartilage tissue engineering is to develop methods that consistently form hyaline cartilage and can be applied with arthroscopic techniques. The present study alms to Investigate the cartilage regenerative response of an injectable fibrin based hyperviscous chondrocyte suspension in a goat femoral condyle full thickness cartilage defect model.

### Methods

10 adult goats were used for the study. 6 mm circular defect was created in both medial femoral condyles. Cartilage tissue was harvested for chondrocyte culture. At secondary open surgery the defects were randomized to the following two treatment groups. 1. Microfracture (1 mm punctures through subchondral bone) (control) 2. Fibrin/chondrocyte paste in the defect. Animals were followed for 4 month. Analyses: ICRS macroscopic scoring (0-12). Mechanical test was performed to assess stiffness of regeneration tissue. Histological analyses was performed by O, Driscoll and Pinada cartilage scores and percentage filling of the defects.

### Results

The ICRS score demonstrated equal scores between groups. The mechanical test demonstrated no difference between treatment groups. However both groups were significantly stiffer than uninjured cartilage. Histology demonstrated limited regeneration response in both groups. In the microfracture group subchondral bone changes with cysts were observed. Cartilage scores and tissue fill was equal in both groups.

### Conclusion

Macroscopic and histologic scoring demonstrated that microfracture and fibrin/chondrocytes stimulated a limited cartilage regeneration response. Mechanical testing revealed increased stiffness of all treatment groups compared to normal cartilage indicating thin layers of cartilage repair tissue resulting in the subchondral bone to contribute to the tissue stiffness. In conclusion a hyperviscous fibrin based chondrocyte suspension did not stimulate cartilage repair better than microfracture. This can probably be explained by insufficient cell containment and cell support compared to other cartilage tissue engineering methods as well as a possible loss of the injected material due to stress from the animal.

1-60. (canceled)

61. A kit of parts, for the treatment of defects in living mammalian tissue by transplantation of living mammalian cells, said kit comprising a first component comprising a biocompatible scaffold, and a second component comprising mammalian cells and a biologically acceptable fixative precursor; wherein, said first component and said second component are isolated from one another.

62. The kit of parts according to claim 61, wherein the mammalian cells are in the form of a cell suspension in a medium, wherein the biologically acceptable fixative precursor and the cell suspension are kept separately from one another.

63. The kit of parts according to claim 62, wherein the mammalian cells and a biologically acceptable fixative precursor of the second component are mixed prior to use.

64. The kit of parts according to claim 61, wherein the second component comprises a mixture of the mammalian cells and the biologically acceptable fixative precursor

65. The kit of parts according to claim 61, which comprises a third component comprising a fixative precursor conversion agent is further provided and wherein said conversion agent is either isolated from said first and said second component, or wherein said conversion agent is part of said first component.

66. The kit of parts according to claim 65, wherein said conversion agent is incorporated into said biologically acceptable scaffold.

67. The kit of parts according to claim 65, where the conversion agent is a cross-linking agent.

68. The kit of parts according to claim 65, wherein the conversion agent is a protein or a polysaccharide.

69. The kit of parts according to claim 65, wherein said conversion agent is lyophilized or applied as a solvent, together with said biologically acceptable scaffold.

70. The kit of parts according to claim 61, wherein the fixative is in the form of a hydrogel.

71. The kit of parts according to claim 61, wherein the mammalian cells are immuno-compatible with said living mammalian tissue.

72. The kit of parts according to claim 71, wherein said mammalian cells are obtained or derived from said individual mammal.

73. The kit of parts according to claim 61 wherein the mammalian cells are in the form of a cell suspension or tissue explant.

74. The kit of parts according to claim 61, wherein the mammalian cells are autologous, homologus (allogenic) or xenogenic in origin

75. The kit of parts according to claim 61, wherein the mammalian cells originate from multipotent or pluripotent stem cells.

76. The kit of parts according to claim 61, wherein the mammalian cells are selected from the group consisting of: fibroblasts, skin cells, keratinocytes, chondrocytes, endothelial cells, chondrocytes, osteoblasts and periodontal cells.

77. The kit of parts according to claim 61, wherein the cells in the second component are present in a sufficient amount of cells to result in regeneration or repair of the target tissue or defect, such as of about 0.1×10⁴ cells/ml to about 10×10⁶ cells/ml.

78. The kit of parts according to claim 61, wherein said biocompatible scaffold is hydrophilic and/or is prepared prior to insertion onto the site of defect by the application of a biocompatible wetting agent.

79. The kit of parts according to claim 78 wherein the scaffold is porous to water and/or an isotonic buffer.

80. The kit of parts according to claim 78, wherein the scaffold essentially consists or comprises a polymer of molecular weight greater than about 1 kDa, such as between about 1 kDa and about 1 million kDa, such as between 25 kDa and 75 kDa.

81. The kit of parts according to claim 78 wherein the scaffold is in the form selected from the group consisting of: a membrane, non-woven and woven fibres, freeze dried polymer such as freeze dried polymer sheets.

82. The kit of parts according to claim 61 wherein the scaffold is synthetic.

83. The kit of parts according to claim 61, wherein a further compound is incorporated into the first and/or second component, such as in the scaffold, wherein the further compound is selected from the group consisting of; hyaluronic acid (HA), hydroxyl apatite (e.g. in the form of granules), growth factors, such as IGF-1, collagen.

84. The kit of parts according to claim 61, wherein the pores of the scaffold are at partly occupied by a component which facilitates the cell adhesion and/or in-growth for regeneration of tissue, such as a component selected from the group consisting of: Chondroitin sulfate, hyaluronan, heparin sulfate, heparan sulfate, dermatan sulfate, growth factors, fibrin, fibronectin, elastin, collagen, gelatin, and aggrecan.

85. The kit of parts according to claim 83, wherein hyaluronic acid is incorporated into the scaffold.

86. The kit of parts according to claim 83, wherein the hyaluronic acid is present in the scaffold at a proportion of between about 0.1 and about 15 wt %.

87. The kit of parts according to claim 61 wherein the scaffold is comprises a compound selected from the group consisting of: polylactide (PLA), polycaprolactone (PCL), polyglycolide (PGA), poly(D,L-lactide-co-glycolide) (PLGA), MPEG-PLGA (methoxypolyethyleneglycol)-poly(D,L-lactide-co-glycolide).

88. The kit of parts according to claim 87, wherein the scaffold consists or comprises PLGA or MPEG-PLGA.

89. The kit of parts according to claim 88, wherein the MPEG-PLGA is a polymer of the general formula:
A-O-—(CHR¹CHR²O)ₙ—B
wherein;
A is a poly(lactide-co-glycolide) residue of a molecular weight of at least 4000 g/mol, the molar ratio of (i) lactide units and (ii) glycolide units in the poly(lactide-co-glycolide) residue being in the range of 80:20 to 10:90,
B is either a poly(lactide-co-glycolide) residue as defined for A or is selected from the group consisting of hydrogen, C₁₋₆-alkyl and hydroxy protecting groups,
one of R¹ and R² within each —(CHR¹CHR²O)— unit is selected from hydrogen and methyl, and the other of R¹ and R² within the same —(CHR¹CHR²O)— unit is hydrogen,
n represents the average number of —(CHR¹CHR²O)— units within a polymer chain and is an integer in the range of 10-1000,
the molar ratio of (iii) polyalkylene glycol units —(CHR¹CHR²O)— to the combined amount of (i) lactide units and (ii) glycolide units in the poly(lactide-co-glycolide) residue(s) is at the most 20:80,
and wherein the molecular weight of the copolymer is at least 10,000 g/mol, preferably at least 15,000 g/mol.

90. The kit of parts according to claim 89, wherein both of R¹ and R² within each unit are hydrogen.

91. The kit of parts according to claim 89, wherein B is a poly(lactide-co-glycolide) residue as defined for A.

92. The kit of parts according to 89, wherein B is C₁₋₆-alkyl.

93. The kit of parts according to 89, wherein B is a hydroxy protecting group.

94. The kit of parts according to 89, wherein B is a hydroxy group.

95. The kit of parts according to claims 87, wherein the scaffold is prepared by freeze drying a solution comprising the compound in solution.

96. The kit of parts according to claims 87, wherein the scaffold has a porosity in the range of 50 to 97%.

97. The kit of parts according to claim 61, where in the biocompatible scaffold comprises a biological polymer, such as protein or polysaccharide.

98. The kit of parts according to claim 97, wherein the biological polymer is selected from the group consisting of: gelatin, collagen, alginate, chitin, chitosan, keratin, silk, cellulose and derivatives thereof, and agarose.

99. The kit of parts according to claim 61, wherein the biologically acceptable fixative precursor is a biologically obtained or derived component, such as fibrinogen.

100. The kit of parts according to claim 99, wherein the fibrinogen is recombinantly prepared.

101. The kit of parts according to claim 99, wherein the fibrinogen is isolated from a mammalian host cell such as a host cell obtained or derived from the same species as the individual mammal, or a transgenic host.

102. The kit of parts according to claim 99, wherein the concentration of fibrinogen used is 1-100 mg/ml.

103. The kit of parts according to claim 61, wherein the conversion agent is selected from the group consisting of: thrombin, a thrombin analogue, recombinant thrombin or a recombinant thrombin analogue.

104. The kit of parts according to claim 103, wherein the concentration of thrombin used is between 0.1 NIH unit and 150NIH units, and/or a suitable level of thrombin for polymerizing 1-100 mg/ml fibrinogen.

105. The kit of parts according to claim 61, which comprises an integrated supply device, comprising the following functionally linked devices: (i) at least one container which contains said second component prior to use, (ii) a force applicator for pressurizing said second component out of said container and into (iii) a connector to (iv) a delivery device, wherein said delivery device is suitable for direct application of the second component to the first component inserted in the site of defect in living mammalian tissue.

106. A kit of parts according to claim 105, wherein said integrated supply device comprises two containers, a first container which contains said cell suspension, and a second container which contains said fixative precursor, wherein said first and second containers are joined by a common connector to allow mixing of said cell suspension and said fixative precursor concomitantly to prepare the second component prior to delivery by said delivery means

107. The kit of parts according to claim 105, wherein said integrated supply device comprises a further container which contains said conversion agent, wherein said further container is joined by a common connector to said first, and optionally said second containers by said common connector to allow mixing of said first component with said conversion agent immediately prior to delivery by said delivery means to said first component inserted in the site of defect in said tissue.

108. The kit of parts according to claim 106, wherein and said two containers and/or further container are functionally linked to a force applicator which may be either a common force applicator device or separate independent force applicators.

109. The kit of parts according to claim 105, wherein said at least one container, such as the first container, the second container and/or said third container, are in the form a syringe body, and said force applicator(s) are in the form of the respective syringe plunger.

110. The kit of parts according to claim 105, wherein said connector is or comprises at least one a tube, which has a proximal end/or ends which is/are connected to said one or more containers, and a single distil end which is connected to said delivery device.

111. The kit of parts according to claim 110, wherein said connector further comprises a mixing device to allow thorough mixing of the second component and optionally said conversion agent prior to entry into said delivery device.

112. The kit of parts according to claim 105, wherein said delivery device is in the form of a medical device selected from the group consisting of: a syringe, a catheter, a needle, and a tube, a spraying device and a pressure gun.

113. The kit of parts according to claim 61, wherein the kit is for the treatment of defects in living mammalian tissue defects in living mammalian tissue wherein the tissue defect is selected from the group consisting of: cartilage defect, bone defect, skin defect, and periodontal defect.

114. The kit of parts according to claim 61, wherein the kit is for use in, a method of surgery, such as a method of endoscopic, atheroscopic, or minimal invasive surgery, and conventional or major open surgery.

115. The kit of parts according to claim 61, wherein the scaffold is in a form selected from the group consisting of; a sheet, a membrane, a molded form, a plug, a tube, a sphere, a three dimensional form prepared for insertion into site of defect, or an implant, a cosmetic implant, a reconstructive implant.

116. The kit of parts according to claim 61, for use in reconstruction surgery or cosmetic surgery.

117. A method for repairing defects in a tissue of a living individual mammal, such as a human being, by transplantation of living mammalian cells, said method comprising:
i) The concomitant application of
A first component comprising a biocompatible scaffold; and
A second component comprising a mixture of mammalian cells and a biologically acceptable fixative precursor
to the site of said defect, prior to the conversion of the fixative precursor into a fixative, and
ii) fixing said mammalian cells to said scaffold, and said scaffold to said living mammalian tissue by conversion of the fixative precursor into a fixative.
Wherein, the first component, the biocompatible scaffold, the second components, the mammalian cells, and the biologically acceptable fixative precursor is as defined in claim 61.

118. The method according to claim 117, wherein said first component is applied to site of said defect, prior to application of said second component.

119. The method according to claim 117, where in said second component is applied to site of said defect either prior to or concurrently as said first component.

120. The method according to claim 117 wherein a third component comprising a fixative precursor conversion agent is concomitantly applied to site of said defect, wherein the conversion agent is as defined in a kit of parts, for the treatment of defects in living mammalian tissue by transplantation of living mammalian cells, said kit comprising a first component comprising a biocompatible scaffold, and a second component comprising mammalian cells and a biologically acceptable fixative precursor; wherein, said first component and said second component are isolated from one another.

121. The method according to claim 120, wherein said conversion agent is applied as part of said first component.

122. The method according to 117, wherein said method is performed in reconstruction surgery or cosmetic surgery.
